(19) United States
(12) Patent Application Publication
(10) Pub. No.: US 2008/0171059 A1
(43) Pub. Date: Jul. 17, 2008

(54) METHODS AND COMPOSITIONS FOR INCREASED PRIMING OF T-CELLS THROUGH CROSS-PRESENTATION OF EXOGENOUS ANTIGENS

(76) Inventors: Shanshan Wu Howland, Cambridge, MA (US); Lloyd Old, New York, NY (US); K. Dane Wittrup, Chestnut Hill, MA (US)

Correspondence Address: BAKER DONELSON BEARMAN CALDWELL & BERKOWITZ, PC, 555 11TH STREET, NW, 6TH FLOOR, WASHINGTON, DC, 20004, US

(21) Appl. No.: 11/834,910

(22) Filed: Aug. 7, 2007

Related U.S. Application Data
(60) Provisional application No. 60/835,873, filed on Aug. 7, 2006.

(51) Int. Cl. A61K 39/00; C12N 5/00; A61P 37/00

(52) U.S. Cl. 424/185.1; 435/375

(57) ABSTRACT

Methods for eliciting in an animal in need thereof a cell-mediated immune response specific to an antigen, the method comprising providing an antigen preparation comprising particles on the surface of which the antigen is attached, and administering the antigen preparation to the animal, wherein the particles are taken up by antigen presenting cells (APC) of the animal via phagocytosis, forming a phagosome inside the APC, wherein the antigen is attached to the surface of the particle in such a way that the antigen is released in the phagosome before the phagosome fuses with a late endosome or a lysosome, and wherein the antigen is cross-presented on a Class I MHC molecule. Also provided are particulate antigen preparations or particulate vaccines that can be delivered to an animal in need thereof for vaccination against, for preventing or treating, a disease related to the antigen, such as cancer and a viral infection.

### CROSS REFERENCE TO RELATED APPLICATIONS

This application claims priority to U.S. Pat. App. No. 60/835,873, the disclosure of which is incorporated herein in its entirety.

### FIELD OF THE INVENTION

This invention relates to compositions and methods for immuno-therapy, specifically, for increasing cross-presentation of exogenous antigens so that cytotoxic or cellular immune response to the antigen in an animal is enhanced.

### BACKGROUND OF THE INVENTION

Vaccines that stimulate antibody production (humoral immunity) have enjoyed success for more than two centuries. Humoral immunity, however, is of limited effectiveness against cancers and certain viral diseases like HIV and herpes simplex virus, because many tumor-associated antigens and viral antigens are intracellular and inaccessible to the antibody. Effective cellular immune responses (cell-mediated immunity), especially cytotoxic T lymphocytes (CTLs), are the best weapons amongst the immune system's arsenal against these diseases.

The development of vaccines that generate effective cellular immune responses, in particular, CD8⁺ cytotoxic lymphocytes, however, remains a challenge, partly because exogenous antigens introduced to the body by vaccines, unlike endogenous antigens (e.g. cancer cells and viral infected cells of the body), have not been effective in eliciting the production of antigen-class I histocompatibility molecules (MHC class I) complexes required to prime CD8⁺ T cells.

In order for the antigen to be recognized by CD8⁺ T cells, it must be complexed with MHC class I molecules. Endogenous antigens almost always are degraded into peptides by proteasomes. The resultant peptides are picked up by TAP (transporter associated with antigen processing), and eventually complexed with MHC class I molecules, displayed on the surface of the cell. The antigen-MHC class I complex is recognized by CD8⁺ cytotoxic T cells.

Exogenous antigens, on the other hand, are taken up by antigen-presenting cells (APCs), such as dendritic cells, by endocytosis or phagocytosis. The endosome or phagosome so formed predominantly fuses with lysosomes, where the antigen is degraded into fragments which are then nestled within a class II histocompatibility molecule (MHC II) and displayed at the surface of the cell, and are recognized by CD4⁺ T cells.

It is known that when a particulate matter of a suitable size with antigen attached thereto enters the body, it is taken up by a suitable APC, and the antigen is released in the phagosome. This release is followed by a phagosome-to-cytosol translocation. Through a so-called cross-presentation process, the mechanism of which is still poorly understood, exogenous antigen may also be displayed in MHC class I molecules. Phagocytosed exogenous antigens somehow escape to the cytosol to be processed by proteasomes and are loaded onto nascent MHC class I molecules, prompting recognition by and activation of CD8⁺ T cells.

The lack of success in the development of vaccines that generate effective cellular immune responses can be explained by the fact that exogenous antigens do not get effectively cross-presented. Although it has been observed that ovalbumin passively adsorbed to latex beads was more efficiently cross-presented than ovalbumin conjugated to the same beads, no explanation was put forth at that time (Kovacsovics-Bankowski et al., 1993), and such observations have not led to the insight that the antigen release kinetics can be manipulated to increase cross-priming of CD8+ T cells. Therefore, there is a need for methods and compositions that increase cross-presentation of exogenous antigens, such that vaccines that bear these exogenous antigens can prime cytotoxic T cells and induce cellular immunity in a patient in need thereof.

Yeast vehicles and their use as antigen delivery system are known in the art, see for example U.S. Pat. No. 5,830,463, which further discloses that the yeast vehicles are capable of stimulating an immune response including the production of cytotoxic T cells to kill cancer cells. However, there has never been any teaching or suggestion in the prior art that the antigen should be presented on the surface of the yeast delivery vehicle, or that the antigen should be released from particles that carry the antigen within a time limit after the particle has been taken up by an antigen presenting cell via phagocytosis.

### SUMMARY OF THE INVENTION

It has now been surprisingly discovered that MHC class I presentation of an exogenous antigen can be increased by controlling the timing of release of the antigen from the surface of a particle that is taken up by an APC. Specifically, it has been surprisingly discovered that if the antigen is released within a time window of about 30 minutes, preferably in less than 25 minutes, after the particle is phagocytosed by a dendritic cell, cross-presentation of the released antigen is maximized and the particle bearing the antigen will be able to cross-prime CD8⁺ T cells, and serve as an effective vaccine for treating diseases related to the antigen.

Accordingly, in one embodiment, a method is provided for eliciting in an animal in need thereof a cell-mediated immune response specific to an antigen, the method comprising (1) providing an antigen preparation comprising a particle having a surface on which the antigen is attached, wherein upon phagocytosis of the particle by an antigen presenting cell, the antigen is released from the particle in a phagosome before the phagosome fuses with a late endosome or a lysosome, and wherein the antigen is cross-presented on a Class I MHC molecule, and (2) administering the antigen preparation to the animal. The animal suitable for treatment may preferably be a mammal, in particular a human.

In one embodiment, the antigen released from the particle has a molecular weight of less than about 500 kDa. The antigen may preferably be a protein or a derivative thereof, such as a cancer antigen. In certain embodiments, the cancer antigen is selected from the group consisting of New York Esophageal 1 antigen (NY-ESO-1), MAGE-A1, MAGE-A2, MAGE-A3, MAGE-A4, MAGE-A6, MAGE-A8, MAGE-A10, MAGE-B, MAGE-C1, MAGE-C2, L antigen (LAGE), synovial sarcoma X breakpoint 2 (SSX2), SSX4, SSX5, preferentially expressed antigen of melanoma (PRAME), Melan-A, Tyrosinase, MAGF, PSA, CEA, HER2/nev, MART1, BCR-abl; and a mutant oncogenic form of p53, ras, myc or RB-1.

In certain embodiments, the particle has a size that allows effective phagocytosis by the APC, such as having a diameter or a cross section that ranges between about 0.3 μm and about 20 μm.

In certain embodiments, the antigen presenting cell is a dendritic cell.

Preferably, the particle is a genetically engineered host cell transformed with an expression vector, and wherein the antigen is a fusion protein encoded by the expression vector, and wherein the fusion protein comprises (1) an antigenic peptide, (2) a signal peptide (or a surface anchor sequence) for anchoring the fusion protein to the surface of the host cell, and (3) a protease recognition site that lies between the antigenic peptide and the surface anchor sequence, wherein the protease recognition site is recognized by a protease in the phagosome to release the antigenic peptide from the host cell surface rapidly inside the phagosome. In certain embodiments, the host cell is a yeast cell, such as the yeast Saccharomyces cerevisiae, particularly the SWH100 and the EBY100 strains described herein below.

In certain preferred embodiments, the surface anchor sequence is a yeast mating adhesion receptor subunit Aga2p. The antigen may be linked to the signal sequence via a (G₄S)₃ linker.

In certain embodiments, the protease recognition site is a Cathepsin S (CatS) recognition site.

In certain embodiments, the particle is a cell, preferably a microbial cell having a wall, on which a fusion protein comprising the antigen is attached via conjugation such via a chemical reaction. Preferably, the microbial cell is a yeast cell, such as a Saccharomyces cerevisiae cell. The protease recognition site is a Cathepsin S (CatS) recognition site. The yeast may be the strain SWH100, preferably rendered non-viable via radiation, or treatment with a chemical agent, or both.

The method according to claim 15, wherein the fusion protein comprises a fusion of a maltose-binding protein, SNAP-tag, 4 repeats of Cathepsin S recognition site EKARVLAEAA, and NY-ESO-1 as the antigen.

In certain embodiments, the fusion protein comprises an amino acid sequence of SEQ ID NO: 1.

In certain embodiments, the particle is a pharmaceutically acceptable preparation of fungal or bacterial cell wall, such as zymosan or a yeast cell wall preparation. In certain embodiments, the particle comprises polymer beads, inorganic particles, micelles or colloidal complexes. The polymer beads may comprise latex beads, poly(lactic-co-glycolic acid) beads, polystyrene beads, or chitosan beads. The inorganic particles may be selected from the group consisting of iron oxide particles, glass beads, silica beads, gold particles, and Quantum Dots™. The particles may comprise Immune-stimulating complexes (ISCOMs), or liposomes.

The present invention further provides a composition comprising a particle having a surface on which an isolated antigen is attached, wherein the antigen is releasable from the particle in a phagosome upon phagocytosis of the particle by an antigen presenting cell, the antigen is released from the particle before the phagosome fuses with a late endosome or a lysosome, and wherein the antigen is cross-presented on a Class I MHC molecule. The term “isolated antigen,” as used in the context of this invention, refers to an antigen that is substantially free from other components with which it is naturally associated. For example, isolated antigens may be purified from a host cell in which they naturally occur, or in which they are genetically engineered to be produced.

In another embodiment, the present invention provides a method for treating a population of cells, a cultured tissue, a cultured organ, or an animal in need thereof, the method comprising contacting said population of cells, cultured tissue or cultured organ of an animal with a pharmaceutical composition comprising a composition of the present invention and a pharmaceutically acceptable excipient. The above treated cells or organs or cultured tissues may be further transferred back to an animal for inducing a desirable cell-mediated immune response against the antigen.

### BRIEF DESCRIPTION OF THE DRAWINGS

Other objects, advantages and novel features of the present invention will become apparent from the following detailed description when considered in conjunction with the accompanying drawings.

FIG. 1 shows that antigen displayed on the surface of yeast is cross-presented. (A) Diagram of the yeast surface display system of strain EBYN9V. The N9V epitope is in bold. (B) Dose response of EBYN9V on cross-presentation. EBYN9V and wild type EBY100 yeast were added to DCs at the indicated ratios. After 24 h, the DCs were assayed for the ability to stimulate IFNγ secretion in co-cultured N9V-specific T cells. Error bars represent the standard deviations of duplicate wells. (C) Amino Acid Sequence of Peptide Surface-Displayed in EBYN9V (SEQ ID NO. 23). (D) Amino Acid Sequence of Peptide Surface-Displayed in EBY(C1)₄N9V (SEQ ID NO: 24).

FIG. 2 shows that surface-displayed antigen is cross-presented more efficiently than antigen expressed intracellularly in yeast. (A) EBYN9V yeast and yeast expressing the same Aga2p-N9V fusion protein intracellularly were added to DCs at a 20:1 ratio and tested for the ability to stimulate IFNγ secretion in co-cultured N9V-specific T cells. Lactacystin (5 μM) or chloroquine (25 μM) were added to some wells an hour before the yeast were introduced. Error bars represent the standard deviations of duplicate wells. (B) Samples of the two yeast cultures and wt yeast were subjected to extensive reduction to release proteins disulfide-bonded to the cell wall. Subsequently, the yeast were treated with Zymolyase and lysed. The proteins reduced off the cell wall and the lysed cell extracts were slot-blotted onto the same nitrocellulose membrane and labeled for c-myc.

FIG. 3 shows a correlation between linker susceptibility to CatS cleavage and cross-presentation efficiency. (A) and (C) Yeast surface-displaying N9V with different linkers (described in Table 1) were incubated with 50 ng (A) or 20 ng (C) of recombinant CatS for 15 min at 37° C. The percentage decrease in c-myc levels in comparison to yeast before CatS treatment was determined by flow cytometry. (B) and (D) The yeast samples with different linkers were added to DCs and assayed for the ability to stimulate IFNγ secretion in co-cultured T cells 24 h later. The results were normalized by the percentage of IFNγ+T cells stimulated by yeast with the unmodified linker (B) or a single insert of the C1 sequence (D). In (B), there were significant differences in surface display levels between constructs, so each yeast culture sample was mixed with wt yeast to normalize the delivered antigen dose. This was unnecessary in (D) as the surface display levels were within 10% of each other. Error bars represent standard deviations between duplicate wells.

FIG. 4 shows a comparison of different linkers, suggesting that the phagosome-to-cytosol route is involved and there is a time window of antigen release for optimal cross presentation. (A) Yeast strains surface-displaying N9V with different linkers were added to DCs and assayed for the ability to stimulate IFNγ secretion in co-cultured T cells 24 h later. The antigen display levels varied by less than 10%. Lactacystin (5 μM) or chloroquine (25 μM) were added to some wells an hour before the yeast were introduced. Error bars represent the standard deviations of duplicate wells. (B) and (C) At various time points after the yeast were added at a 2.5:1 ratio, the DCs were placed on ice and lysed with RIPA buffer. The yeast thus extracted were labeled for the presence of α-hemagglutinin (HA) and c-myc epitopes and analyzed by flow cytometry.

FIG. 5 demonstrates mathematical modeling and experimental results for cross-presentation of antigen attached to yeast cells by scFv binding. (A) Schematic of a simple model describing antigen release, export and degradation before and after phagocytosis. The unbroken arrows represent first-order processes with associated rate constants that make up an ordinary differential equation-based model. (B) A representative plot of cytoplasmic antigen versus k_{off} when the equations were solved with reasonable parameter values (pre-phagocytosis time of 30 min, c₁=4, c₂=50, c₃=100, k_{esc}=0.02 min⁻¹, k_{deg}=0.1 min⁻¹). (C) Fluorescein-conjugated yeast were incubated with culture supernatants containing secreted scFv-antigen fusion proteins, washed, and added to DCs to perform cross-presentation assays. The results of three separate experiments (represented by the three line/marker combinations) were scaled such that unloaded fluorescein-conjugated yeast gave a result of 1 whereas a 1 μM extended peptide positive control gave a result of 100. (D). Fluorescein-conjugated yeast loaded with scFv-antigen were incubated with DCs for 15 min, after which the DCs were placed on ice and lysed with RIPA buffer. The released yeast cells were analyzed for the presence of the c-myc epitope tag by flow cytometry.

FIG. 6 are two examples of model-predicted time course behavior at different scFv dissociation rates. The model ODEs were solved for three hypothetical scFvs with different dissociation half times (t_{1/2}=ln(2)/k_{off}), demonstrating that an intermediate dissociation rate gives rise to the highest final cytoplasmic antigen level and hence the highest cross-presentation efficiency. The amounts of yeast-bound antigen (A) and cytoplasmic antigen (B) vs time were determined with the following parameter values: c₁=4, c₂=50, c₃=100, k_{esc}=0.02 min₋₁, k_{deg}=0.1 min₋₁.

FIG. 7 shows the amino acid sequence and components of fusion protein MSE (SEQ ID NO: 1).

FIG. 8 shows the amino acid and components of fusion protein MSCcmyc (SEQ ID NO: 2).

FIG. 9 shows that yeast conjugated with MSE on the wall is processed by DCs and presented to NY-ESO-1-specific (A) CD8⁺ and (B) CD4⁺ T cell clones. Indicated number of T cells (20,000 or 4,000 for CD8+ T cells, and 1,100 or 400 for CD4+ T cells) were co-cultured with 50,000 antigen-pulsed DCs for 24 hours in these ELISPOT assays.

FIG. 10 shows that yeast conjugated with MBP-ESO on the wall is processed by DCs and presented to NY-ESO-1-specific CD4⁺ T cell clone (B) but not CD8⁺ T cells clone (A). Indicated number of T cells (20,000 or 4,000 for CD8+ T cells, and 1,500 or 300 for CD4+ T cells) were co-cultured with 50,000 antigen-pulsed DCs for 24 hours in these ELISPOT assays.

### DESCRIPTION OF PREFERRED EMBODIMENTS

The present inventors have discovered that the rate of antigen release in the phagosome directly affects the efficiency of antigen cross-presentation occurring via the phagosome-to-cytosol route, with an apparent time window of about 25 to 30 minutes post-phagocytosis for antigen release to be productive in priming CD8+ T cells. Accordingly, in one embodiment, the present invention provides a method for eliciting in an animal, preferably a mammal, in need thereof a cell-mediated immune response specific to an antigen. In one embodiment, the method of the present invention comprising (1) providing an antigen preparation comprising particles on the surface of which the antigen is attached, and (2) administering the antigen preparation to the animal, wherein the particles are taken up by antigen presenting cells (APC), preferably dendritic cells, via phagocytosis, forming a phagosome inside the APC, wherein the antigen is attached to the surface of the particle in such a way that the antigen is released in the phagosome before the phagosome fuses with a late endosome or a lysosome, and wherein the antigen is cross-presented on a Class I MHC molecule.

The present invention further provides for particular antigen preparations that are suitable for use with the method above. The method and compositions of the present invention can be used for treating or preventing cancer, as cancer vaccines, and for preventing and treating certain viral infections where the cellular immune response is required or necessary. Particulate vaccines have advantages over soluble vaccines in that they are not diluted by diffusion, and are targeted to phagocytic professional antigen-presenting cells.

The antigen preparation or vaccines of the present invention may be used in vivo, i.e. via direct administration to an animal, especially a mammal, such as a human, e.g. via injection or other administration routes well-known to those skilled in the art. The antigen preparation or vaccines of the present invention may also be used ex vivo. Instead of injecting the vaccine into the body of the animal, APCs may be first obtained from the animal, and treated with the vaccine ex vivo. The treated APCs are then placed back into the body, which will stimulate the animal's T cells in vivo.

In one embodiment, the present invention provides methods for delivering the antigen preparation of the present invention to an animal or to cells in culture. Such compositions can be delivered to an animal either in vivo or ex vivo, or can be delivered to cells in vitro. In vivo delivery, as used in the context of the present invention, refers to the administration of an antigen preparation directly to an animal. Such administration can be systemic, mucosal and/or proximal to the location of the targeted cell type. Examples of direct administration routes in vivo include aural, bronchial, genital, inhalatory, nasal, ocular, oral, parenteral, rectal, topical, transdermal and urethral routes. Aural delivery can include ear drops, nasal delivery can include nose drops and ocular delivery can include eye drops. Oral delivery can include solids and liquids that can be taken through the mouth. Parenteral delivery can include intradermal, intramuscular, intraperitoneal, intrapleural, intrapulmonary, intravenous, subcutaneous, atrial catheter and venal catheter routes. Oral delivery is useful in the development of mucosal immunity. The antigen preparation of the present invention can be easily prepared for oral delivery, for example, as tablets or capsules, as well as being formulated into food and beverage products. Other routes of administration that modulate mucosal immunity are also preferred, particularly in the treatment of viral infections, epithelial cancers, immunosuppressive disorders and other diseases affecting the epithelial region. Such routes include bronchial, intradermal, intramuscular, nasal, other inhalatory, rectal, subcutaneous, topical, transdermal, vaginal and urethral routes.

In order to administer a yeast vehicle to an organism, dried compositions can be used for oral delivery. The particulate antigen preparation of the present invention can also be mixed with a pharmaceutically acceptable excipient, such as an isotonic buffer that is tolerated by the animal to be treated. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise, for example, dextrose, human serum albumin, and/or preservatives to which sterile water or saline can be added prior to administration.

Ex vivo delivery generally refers to treating a population of cells removed from an animal with the antigen preparation of the present invention under conditions such that the antigen-containing particles are taken up by cells which are then returned to the animal, which will stimulate the animal's T cells in vivo.

In vitro delivery of the antigen preparation of the present invention generally involves treating a population of cells, or even tissues or organs in culture. Cells that are treated in vitro can be maintained in culture or transferred to an animal. It is also possible to directly stimulate a patient's T cells with vaccine-treated APCs outside the body, so as to expand antigen-specific T cells, and then either transplant the T cells back into the body or use the T cells for research purposes.

It is recognized that many suitable particulate preparations are suitable for the present invention. In generally, a particle within a size limit of about 0.3 microns (see e.g. Green et al., 1998, Polyethylene particles of a “critical size” are necessary for the induction of cytokines by macrophages in vitro. Biomaterials 19, 2297-2302) and about 20 microns (see e.g. Cannon et al., 1992, The macrophage capacity for phagocytosis. J. Cell Sci. 101, 907-913) can be effectively and efficiently phagocytosed by an APC, especially a dendritic cell.

Many such particles are available to those of ordinary skills in the art for the preparation of particulate vaccines of the present invention. These include polymer particles such as latex particles, inorganic particles e.g. iron oxide particles, glass beads, silica beads, gold particles, Quantum Dots™; antigen/antibody complexes (multivalent large agglomerates); micelles and colloidal complexes, e.g. Immune-stimulating complexes (ISCOMs); and liposomes.

In a preferred embodiment, particles for the present invention are cells, especially microbial cells, genetically engineered to express an antigen molecule on its surface, including on the surface of the cell wall of the cell, or on the outer membrane of the cell. Preferably, the microbial cell is a yeast cell, in particular a Saccharomyces ceresiviae cell.

Particles for the present invention may also be fragments of a host cell, including but not limited to fragments of cell walls or membranes.

Many methods of attaching the antigen to the particles are also known and available to those skilled in the art. When the particles are a cell, e.g. a bacterial or a yeast host cell expressing the antigen, the antigen can be displayed on the surface of the cell. Display of heterologous peptides or proteins on the surface of recombinant host cells, such as yeast, fungi, mammalian, plant, and bacterial cells are well-established and known in the art. In general, this is accomplished via the targeting and anchoring of the heterologous peptides to the outer surface of a host-cell. See e.g. WO 94/18830 and U.S. Pat. No. 7,169,383. Several methods of displaying protein/peptide antigens on bacterial cell surface display have been developed (see e.g. U.S. Pat. Nos. 5,866,344, 6,190,662, and Georgiou, et al., (1997) Nat. Biotechnol. 15, 29-34.). Similarly, many methods are known in the art for displaying protein/peptide antigens on a yeast cell (see e.g. U.S. Pat. No. 6,696,251; Wittrup, 2001, Protein engineering by cell-surface display. Curr Opin Biotechnol 12:395-399.). The surface-displayed peptides may be released using one or more of a variety of methods, such as via cleavage by a protease, which will be described in more detail below.

The host cell may be a genetically modified microorganism that expresses a surface-displayed protein which binds the antigen or a moiety attached to the antigen. For example, an antibody fragment that binds to fluorescein can be surface-displayed on a yeast cell, and the antigen of interest may be labeled with fluorescein. The fluorescein-labeled antigen can then be bound to the surface of the yeast cell and be cross-presented.

The antigens may also be chemically conjugated to a particle, such as an inactivated nonviable cell or a fragment thereof. The inclusion of a labile disulfide bond or a protease-cleavable site should enhance antigen release in the phagosome. In addition, the use of dendrimers to attach antigen molecules to the yeast cell wall could increase the amount of antigen delivered per cell. Fusion proteins comprising the antigen of interest may be construed that has the ability to bind to some component of the cell wall. For example, chitin-binding domain or lectins can be used to attach the fusion protein to the yeast cell wall. In each of the examples above, it is understood that cell walls (“ghosts” devoid of cytoplasm) or cell wall fragments can be used instead of whole yeast cells. This could reduce delivery of irrelevant yeast proteins and increase the uptake of antigen per dendritic cell.

Several additional attachment methods are available and known to those skilled in the art. These methods are useful when the antigen is to be attached to a particle other than a host cell, or when the antigen of interest is poorly expressed in a recombinant host cell. These attachment methods may be broadly classified into covalent and non-covalent methods.

Covalent methods for attaching the antigen to the particle may further be divided into direct binding and indirect covalent binding. As the name indicates, the direct method conjugates the antigen directly to a chemical group on the particle. In general, chemical methods to directly conjugate a protein antigen covalently to a particle tend to be non-site-specific and often result in some amino acid residues of the antigen being covalently bonded to the particle. This may impede antigen release in the APC phagosome. In the case of particles containing thiol-reactive groups, to avoid this problem, the antigen may be engineered to have an unpaired cysteine residue in a polypeptide region flanking the antigen, for attachment to the particle.

In contrast to direct conjugation, an indirect binding method uses an intermediate, or a tag, which is covalently bound to the particle. The antigen of interest is linked to the tag, e.g. as part of a fusion protein, or via other covalent or non-covalent binding. Indirect binding accordingly can be site-specific. The release of the antigen may be controlled or facilitated via including a protease-sensitive linker or other cleavage moiety between the tag and the antigen.

Many such tags for site-specific conjugation are known in the art, such as the SNAP-tag, HaloTag, C-terminal LPXTG tag, Biotin acceptor peptide, and the peptidyl carrier protein (PCP) or ybbR tag. For example, (1) the SNAP-tag™ (Covalys Biosciences AG, Witterswil, Switzerland) is an engineered O6-alkylguanine-DNA alkyltransferase that forms a covalent bond with benzylguanine derivatives (Keppler et al., 2004, Labeling of fusion proteins of 06-alkylguanine-DNA alkyltransferase with small molecules in vivo and in vitro. Methods 32, 437-444.) that can be conjugated to particle surfaces. A variety of 06-benzylguanine derivatives with different functional groups are commercially available, such as succinimidyl ester for modifying amine-containing particles. HaloTag™ (Promega, Madison, Wis.), is a mutant haloalkane dehalogenase that forms a covalent bond with an alkylchloride group (Los and Wood, 2007, Methods Mol Biol 356, 195-208) that can be conjugated to particle surfaces. A variety of alkylchloride derivatives with different functional groups are commercially available, such as a succinimidyl ester for modifying amine-containing particles. A C-terminal LPXTG tag is recognized by the enzyme Sortase A, which ligates it to triglycine (Parthasarathy et al., 2007, Bioconjug Chem 18, 469-476.). This reference includes a protocol for conjugating first triglycine and then LPETG-tagged protein to amine-terminated microbeads. The biotin acceptor peptide (Chen et al., 2005, Nat Methods 2, 99-104) is a peptide that the E. coli enzyme BirA biotinylates either during expression (by co-expressing BirA) or in vitro. The biotinylated fusion protein containing the antigen can then be attached essentially permanently to streptavidin-coated particles. The peptidyl carrier protein (PCP) or ybbR tag, is covalently modified by the enzyme Sfp phosphopantetheinyl transferase with Coenzyme A derivatives (Yin et al., 2006. Site-specific protein labeling by Sfp phosphopantetheinyl transferase. Nat. Protoc., 1:280-5.). Biotin-Coenzyme A can be synthesized as described by Yin et al. and covalently attached to the PCP or ybbR tag fused to the antigen. Thus biotinylated, the fusion protein can be attached to streptavidin-coated particles. Alternatively, Coenzyme A can be attached to the surface of the particle using a bifunctional linker (such as maleimide-polyethylene glycol-succinimidyl carboxymethyl from Laysan Bio, Arab, Ala.), and subsequently the particle can be incubated with PCP- or ybbR-tagged antigen in the presence of Sfp phosphopantetheinyl transferase.

Non-covalent methods include the use of antibody-antigen (other than the antigen of interest) or other non-covalent protein-ligand interactions for attachment. Preferably, the antibody-antigen interactions are strong enough so as not to detach from the particle prematurely during storage or in the body. For example, a fusion protein can be made of the antigen of interest and a fluorescein-binding antibody fragment with femtomolar affinity (Boder et. al, 2000). The fusion protein may then be attached, non-covalently, to a fluorescein-derivatized particle.

Another non-covalent attachment method is to use biotin and streptavidin. Biotin may be conjugated to a fusion protein containing the antigen, and then attached to a streptavidin-derivatized particle.

The kinetics of antigen release from the particle after phagocytosis may be manipulated, as the binding interaction could be engineered to be pH-sensitive (dissociating in the slightly acidic phagosome) or more simply, protease-susceptible linkers could be inserted into the antibody such that it is destroyed in the phagosome.

Non-site specific chemical conjugation can also be used to attach a “handle” (e.g. fluorescein) or antibody such that the antigen can subsequently be non-covalently associated with the particle. A list of commonly used conjugation schemes (Pierce Chemical) is shown below.

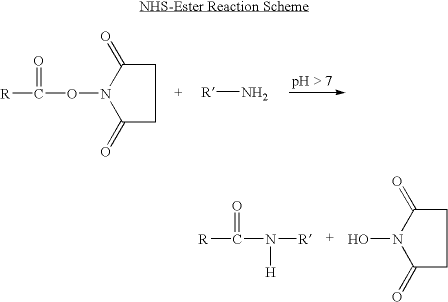

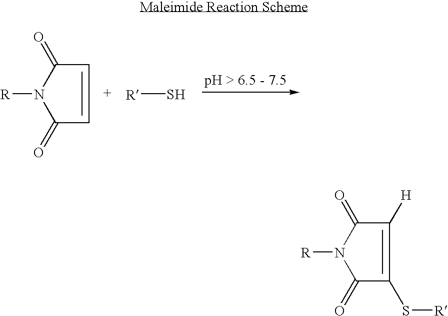

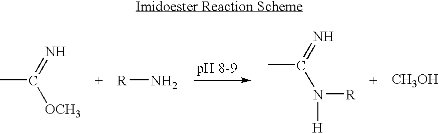

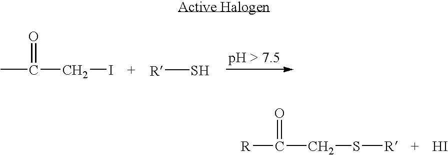

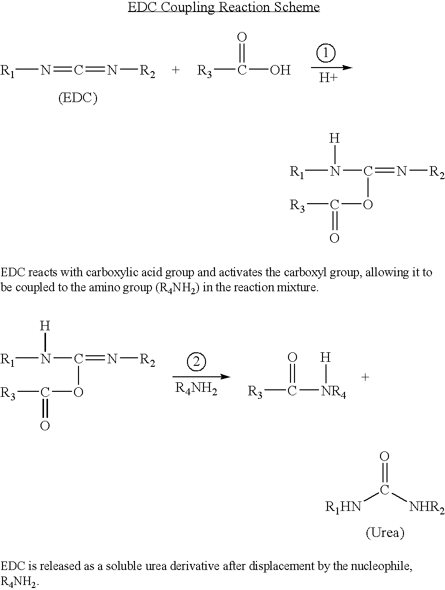

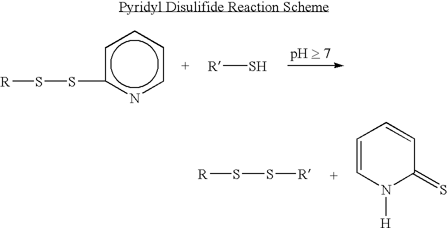

Many companies sell beads and particles that are derivatized with reactive groups in this list.

Similarly, many methods are available for the release of the particle-bound antigen to be timely released. For example, a protease recognition site, e.g. Cathepsin S sites, may be included flanking the antigen, and the antigen will be released from the particle in the phagosome by a protease that recognizes the site. Antibodies or other non-covalent polypeptide binders that bind the antigen with suitable dissociation kinetics may also be used. A pH-sensitive chemical linker, e.g. certain ester, hydrazone, anhydride bonds, may also be used. Similarly, pH-sensitive polymers e.g. those disclosed in Shenoy et al., 2005, Poly(ethylene oxide)-Modified Poly(beta-amino ester) Nanoparticles as a pH-Sensitive System for Tumor-Targeted Delivery of Hydrophobic Drugs. 1. In Vitro Evaluations. Mol Pharm 2, 357-366; Kohane et al., 2003, pH-Triggered Release of Macromolecules from Spray-Dried Polymethacrylate Microparticles. Pharmaceutical Research 20, 1533-1538; Zhang et al., 2004, Synthesis and characterization of partially biodegradable, temperature and pH sensitive Dex-MA/PNIPAAm hydrogels. Biomaterials 25, 4719-4730. In another embodiment, complexing agents may also be used that reverse charge at slightly acidic pH, thus causing the complex to fall apart in the phagosome within the requisite time window. In another embodiment, pH- or temperature-sensitive self-cleaving inteins may be employed as a release mechanism. For example, an intein sequence disclosed in Wood et al., 2000, Biotechnol Prog 16, 1055-1063 cleaves at its C-terminus spontaneously at pH 6, 37° C. but is fairly stable at slightly basic pH and 4° C. Preferably, the intein sequence should have a desirable pH and temperature kinetics, that is, fast cleavage at the acidic pH inside a phagosome but slow cleavage at pH>7). Also, the intein should be protease-resistant.

In preferred embodiments, the antigen of interest is anchored to a particle, especially a cell, via a linker that comprises a Cathepsin S cleavage site. Cathepsin S is a preferred protease for antigen release in the phagosome and for cross-presentation, because it is known that it is highly active early in phagosomal maturation, and there is evidence that it preferentially accumulates in the phagosomes of dendritic cells (Lennon-Dumenil et al., 2002, Analysis of Protease Activity in Live Antigen-presenting Cells Shows Regulation of the Phagosomal Proteolytic Contents During Dendritic Cell Activation. J Exp Med 196, 529-540).

Cathepsin S is known to recognize and cleave at numerous proteolytic sites (see e.g. Ruckrich et al., 2006, Specificity of human cathepsin S determined by processing of peptide substrates and MHC class II-associated invariant chain. Biol Chem 387, 1503-1511). In some embodiments, the proteolytic site comprises the amino acid sequence EKARVLAEAA (SEQ ID NO:3). The present inventors have recently discovered that the N-terminus of NY-ESO-1 (MQ|AE . . . ) and the cytomegalovirus epitope (NLVPMVA|TV) (SEQ ID No. 4) are cleaved rapidly by Cathepsin S (at the positions indicated by the vertical bars). These sites are also suitable as a Cathepsin recognition and cleavage site.

Many antigens are suitable targets of the particulate vaccines of the present invention. These include antigens that are related to or derived from cancer, or infections by a virus, a fungus, a bacterium, or a parasite.

Cancer antigens suitable for the present invention include but are not limited to the New York Esophageal 1 antigen (NY-ESO-1), many melanoma

TABLE 1
List of CT Antigens

| CT id. | Gene symbol | PMID | Pub. date | Journal |
|---|---|---|---|---|
| CT45 | RP13-36C9.1 | 15905330 | May 19, 2005 | PNAS |
| CT46 | HORMAD1 | 15999985 | Jul. 7, 2005 | Cancer Immun |
| CT47 | RP6-166C19.1 | 12477932 | Dec. 11, 2002 | PNAS |
| CT48 | SLCO6A1 | 15546177 | Nov. 17, 2004 | Cancer Immun |
| CT49 | TAG | 14871852 | Feb. 1, 2004 | Cancer Research |
| CT50 | LEMD1 | 15254688 | Aug. 12, 2004 | Oncol Rep |
| CT51 | HSPB9 | 15503857 | Aug. 1, 2004 | Eur J Cell Biol |
| CT52 | KM-HN-1 | 15447989 | Sep. 15, 2004 | Clin Cancer Res |
| CT53 | ZNF165 | 15354214 | Oct. 18, 2004 | Br J Cancer |
| CT54 | SPACA3 | 15475442 | Oct. 1, 2004 | Clin Cancer Res |
| CT55 | CXorf48 | 15499389 | Oct. 1, 2004 | Biochem |
| | | | | Cell Biol |
| CT56 | THEG | 15905330 | Mar. 31, 2005 | PNAS |
| CT57 | ACTL8 | 15905330 | Mar. 31, 2005 | PNAS |
| CT58 | NALP4 | 15905330 | Mar. 31, 2005 | PNAS |
| CT59 | COX6B2 | 15905330 | Mar. 31, 2005 | PNAS |
| CT60 | LOC348120 | 15905330 | Mar. 31, 2005 | PNAS |
| CT61 | CCDC33 | 15905330 | Mar. 31, 2005 | PNAS |
| CT62 | LOC196993 | 15905330 | Mar. 31, 2005 | PNAS |
| CT63 | PASD1 | 15905330 | Mar. 31, 2005 | PNAS |
| CT64 | NA | 15905330 | Mar. 31, 2005 | PNAS |
| CT65 | TULP2 | 15905330 | Mar. 31, 2005 | PNAS |
| CT66 | NA | 15905330 | Mar. 31, 2005 | PNAS |
| CT67 | Klkbl4 | 15905330 | Mar. 31, 2005 | PNAS |
| CT68 | MGC27016 | 15905330 | Mar. 31, 2005 | PNAS |
| CT69 | NA | 15905330 | Mar. 31, 2005 | PNAS |
| CT70 | NA | 15905330 | Mar. 31, 2005 | PNAS |
| CT71 | SPINLW1 | 15905330 | Mar. 31, 2005 | PNAS |
| CT72 | TSSK6 | 15905330 | Mar. 31, 2005 | PNAS |
| CT73 | ADAM29 | 15905330 | Mar. 31, 2005 | PNAS |
| CT74 | CCDC36 | 15905330 | Mar. 31, 2005 | PNAS |
| CT75/ | NA | 15999985 | Jul. 7, 2005 | Cancer Immun |
| CT46? | | | | |
| CT76 | SYCE1 | 15999985 | Jul. 7, 2005 | Cancer Immun |
| CT77 | CPXCR1 | 15999985 | Jul. 7, 2005 | Cancer Immun |
| CT78 | TSPY1 | 16106251 | Aug. 22, 2005 | Br J Cancer |
| CT79 | TSGA2 | 16120156 | Sep. 1, 2005 | Br J Dermatol |
| CT80 | PIWIL2 | 16377660 | Dec. 23, 2005 | Hum Mol Genet |
| CT81 | ARMC3 | 16397042 | Jan. 1, 2006 | Clin Cancer Res |

antigen (MAGE) such as MAGE-A1, MAGE-A2, MAGE-A3, MAGE-A4, MAGE-A6, MAGE-A8, MAGE-A10, MAGE-B, MAGE-C1, and MAGE-C2, L antigen (LAGE), synovial sarcoma X breakpoint 2 (SSX2), SSX4, SSX5, preferentially expressed antigen of melanoma (PRAME), Melan-A, Tyrosinase, MAGF, PSA, CEA, HER2/nev, MART1, BCR-abl, and mutant oncogenic forms of p53, ras, myc and RB-1. Table 1 below lists additional cancer/testis antigens that are suitable targets of the present invention.

Many viral antigens are also suitable targets of the method and particulate vaccines of the present invention. Examples of viral antigens suitable for the present invention include, but are not limited to, env, gag, rev, tar, tat, nucleocapsid proteins and reverse transcriptase from immunodeficiency viruses (e.g., HIV, FIV); HBV surface antigen and core antigen; HCV antigens; influenza nucleocapsid proteins; parainfluenza nucleocapsid proteins; human papilloma type 16 E6 and E7 proteins; Epstein-Barr virus LMP-1, LMP-2 and EBNA-2; herpes LAA and glycoprotein D; CMV pp 65; as well as similar proteins from other viruses.

The following examples are intended to illustrate preferred embodiments of the invention and should not be interpreted to limit the scope of the invention as defined in the claims.

### EXAMPLES

### Example 1

### Yeast Surface-Displayed Antigen is Cross-Presented to CD8⁺ T Cells

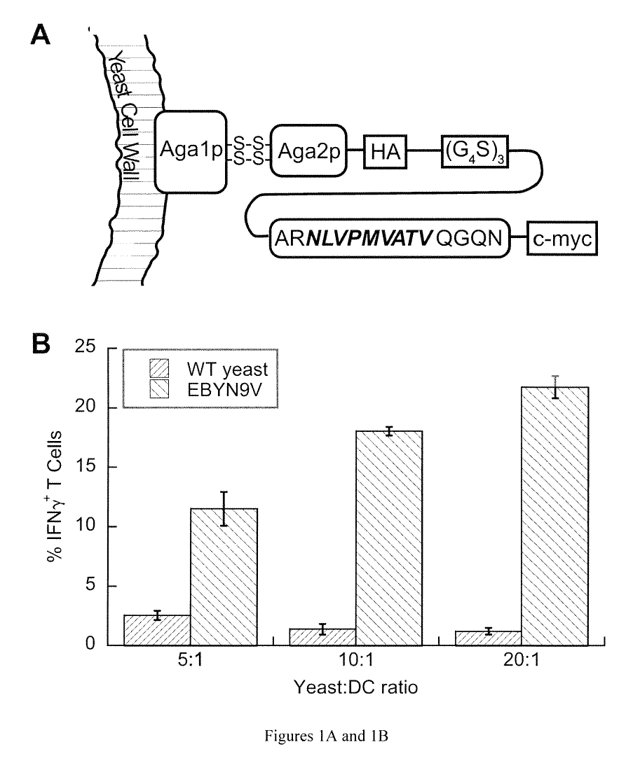

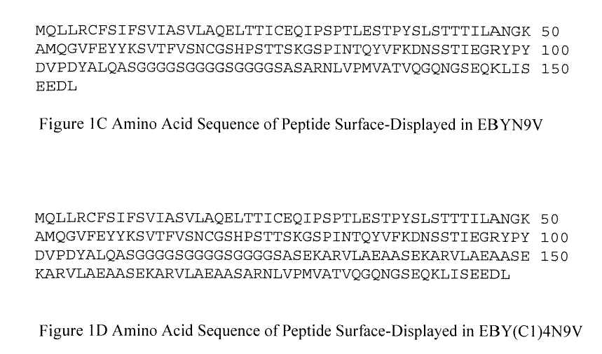

We selected the well-characterized HLA-A*0201-restricted peptide NLVPMVATV (N9V) (SEQ ID NO: 4), derived from cytomegalovirus (CMV) phosphoprotein pp 65 as our model antigen, for which cognate CD8⁺ T cells are available commercially. To ensure proper antigen processing, we included its native flanking sequences in the yeast surface display construct, in the form of the 15-mer ARNLVPMVATVQGQN (SEQ ID NO: 5) that was consistently immunogenic in HLA-A*0201, CMV-positive individuals (Trivedi et al., 2005). The yeast surface display construct consisted of a fusion of this extended peptide to the yeast mating adhesion receptor subunit Aga2p via a (G₄S)₃ linker, with HA and c-myc epitope tags for detection purposes (FIG. 1A). We created the yeast strain EBYN9V with co-inducible chromosomal copies of this construct and Aga1p, with expression resulting in ˜120,000 copies/cell of the Aga2p-N9V fusion anchored to the yeast cell wall by disulfide bonds. The amino acid sequence of the protein that is surface-displayed in EBYN9V yeast is shown in FIG. 1C, which does not contain the Cathepsin S site. The amino acid sequence of a peptide for EBY(C₁)₄N9V (containing a Cathepsin S site) is shown in FIG. 1D (see Examples 3-5 below).

To test for cross-presentation, EBYN9V yeast were added to HLA-A*0201 monocyte-derived DCs at various ratios. The DCs avidly phagocytosed the yeast with an average maximum “capacity” of about 20 yeast per DC (numbers of unphagocytosed yeast rose sharply at higher ratios). Twenty four hours later, the DCs were co-cultured for four hours with a CD8⁺ T cell line specifically recognizing the N9V/HLA-A*0201 complex. An interferon gamma (IFN_{Y}) secretion cell capture FACS assay was performed on the T cells to quantify the percentage of cells that had been activated as a result of cross-presentation by the DCs. As shown in FIG. 1B, EBYN9V yeast resulted in dose-dependent cross-presentation at levels much higher than the background caused by EBY100 yeast lacking the N9V surface display construct. We decided to use the 20:1 yeast:DC ratio for future cross-presentation experiments; note that the concentration of peptide equivalents at this dose is only 4 nM.

### Example 2

### Surface-Displayed Antigen is Cross-Presented More Efficiently than Intracellular Antigen

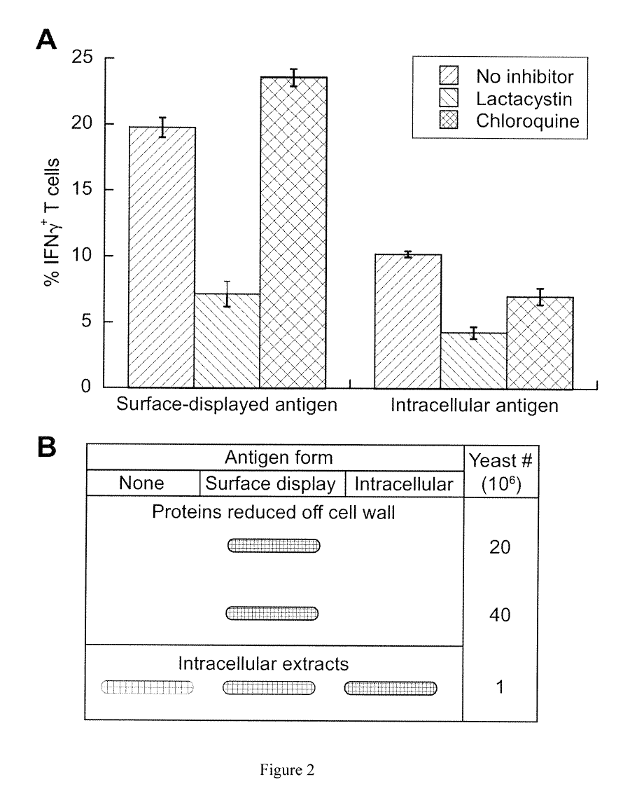

We next compared cross-presentation of yeast surface-displayed antigen to antigen expressed inside the cytosol of yeast. By deleting the surface anchor sequence, the same Aga2p-N9V fusion protein was expressed intracellularly in yeast. At the same 20:1 yeast:DC ratio, cross-presentation resulting from intracellular antigen was only half that from surface-displayed antigen (FIG. 2A). This result was obtained even though the expression level of intracellular antigen was 20-30× the surface display level, as shown by the slot blot in FIG. 2B.

To try to understand the marked difference in cross-presentation efficiency between surface-displayed and intracellular antigens, we studied the effects of inhibitors of either the phagosome-to-cytosol route or the vacuolar route. Cross-presentation of surface-displayed antigen was strongly inhibited by lactacystin, a proteasome inhibitor, whereas chloroquine, which raises the endolysosomal pH, had no inhibitory effect and was actually slightly beneficial (FIG. 2A). We deduced that the phagosome-to-cytosol pathway is the major mechanism of cross-presentation with yeast surface-displayed antigen. Chloroquine has been observed to increase the cross-presentation efficiency of soluble antigens, possibly because it increases membrane permeability and hence antigen escape into the cytosol (Accapezzato et al., 2005), and may be having a similar subtle effect here. Cross-presentation of intracellular antigen was inhibited by both lactacystin and chloroquine (FIG. 2A). It is unclear whether cross-presentation of intracellular antigen proceeds by a combination of the phagosome-to-cytosol and vacuolar routes, or whether only the phagosome-to-cytosol route is involved, with chloroquine reducing the rate at which the yeast cell wall was breached, thus slowing antigen export into the DC cytosol. In any case, it is clear to us that having antigen exposed on the yeast surface provides a significant advantage for cross-presentation due to greater accessibility to the DC cytosol compared to having antigen trapped by the thick yeast cell wall.

### Example 3

### Faster Antigen Release Within the Phagosome Results in More Efficient Cross-Presentation

The rate at which antigen is released from a phagocytosed particle influences the efficiency of cross-presentation, since antigen release is a necessary step before export into the cytosol can occur. With EBYN9V yeast, the N9V antigenic peptide could be released from the yeast cell wall by proteolysis in the phagosome or by reduction of the disulfide bonds tethering Aga2p to Agalp. The rate of the former mechanism could potentially be manipulated by including protease recognition sites N-terminal to the antigenic peptide. We targeted Cathepsin S (CatS) because unlike most other cathepsins that are active only in acidic conditions found later in phagosomal maturation, its operating range extends from pH 5.0 to 7.5 (Pillay et al., 2002). Furthermore, phagosomes in macrophages and DCs fuse preferentially with endocytic compartments enriched in CatS, with CatS activity detected in ten-minute-old phagosomes (Lennon-Dumenil et al., 2002).

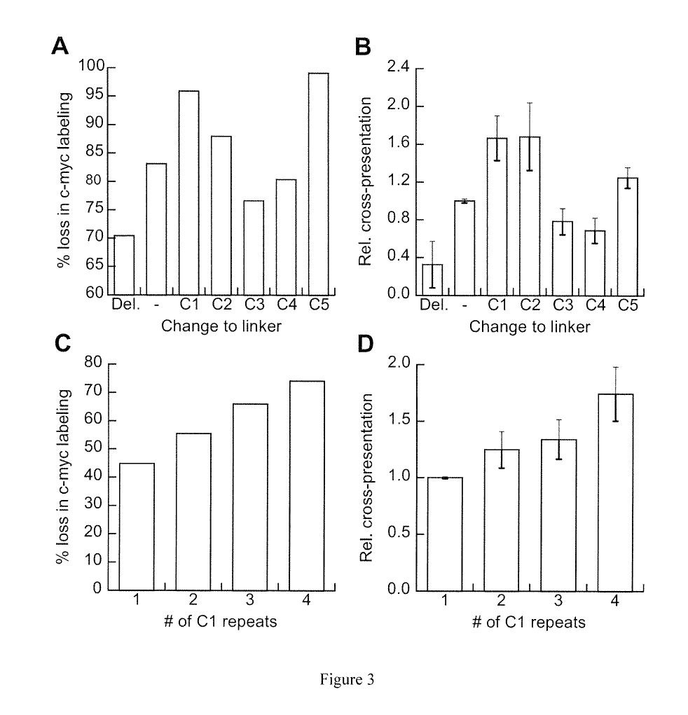

Five known recognition sites are listed in Table 2. In some cases, four amino acid residues on either side of a known CatS cleavage point were used. These sequences, termed C1 to C5, were each inserted individually between the (G₄S)₃ linker and the extended antigenic peptide. An additional construct was created where the (G₄S)₃ linker, a suspected CatS cleavage site, was deleted. To test whether these sequences were recognized in their new context, yeast expressing the modified plasmid constructs were incubated with recombinant CatS and analyzed for loss of the c-myc epitope. CatS had negligible effect on HA epitope levels, indicating that the polypeptide chains linking together HA, Aga2p, Agalp and the cell wall remained intact. While the addition of C1, C2, and C5 increased CatS cleavage, C3 and C4 had the opposite effect and were apparently not recognized and/or disrupted a pre-existing recognition site (FIG. 3A). Deleting the (G₄S)₃ linker altogether conferred the greatest resistance to CatS cleavage. When yeast with these different linker sequences were phagocytosed by DCs, the resulting pattern of cross-presentation was strikingly similar to the pattern of CatS cleavage (FIG. 3B). By performing Spearman's rank correlation on the rankings listed in Table 2, CatS susceptibility and cross-presentation efficiency were found to be positively correlated at the significance level of p<0.05, demonstrating hat faster antigen release within the phagosome results in more efficient cross-presentation.

In an attempt to further increase antigen release rates by CatS, we created constructs with tandem repeats of C1 and C2 sequences. Tandem repeats of C2 did not further enhance CatS susceptibility (not shown), but the rate of CatS cleavage increased with the number of tandem copies of C1 (FIG. 3C), and there was a corresponding increase in cross-presentation efficiency (FIG. 3D).

TABLE 2
Linker Effects on Cross-Presentation

| | | | CatS | Cross- |
|---|---|---|---|---|
| | | | cleavage | presentation |
| Name | Modification | Reference | rank order | rank order |
| Deleted | (G₄S)₃ deleted | — | 7 | 7 |
| Unchanged | — | — | 4 | 4 |
| C1 | EKARVLAEAA | (Thurmond et al., 2004) | 2 | 1 |
| | inserted | | | |
| C2 | SSAESLK inserted | (Zaliauskiene et al., 2002) | 3 | 2 |
| C3 | NWVCAAKF inserted | (Pluger et al., 2002) | 6 | 5 |
| C4 | GILQINSR inserted | (Pluger et al., 2002) | 5 | 6 |
| C5 | QWLGAPVP inserted | (Baumgrass et al., 1997) | 1 | 3 |

### Example 4

### Antigen Released by CatS is Processed by Proteasomes

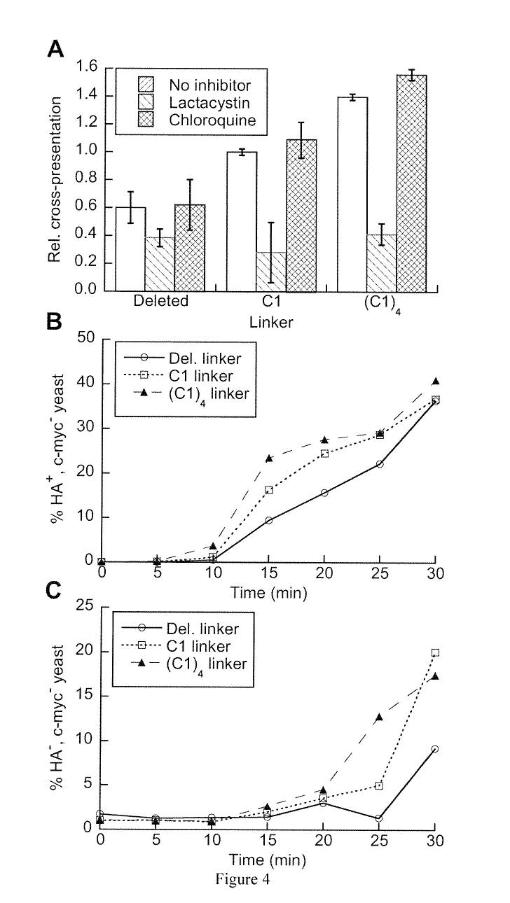

Yeast strains were prepared with chromosomally integrated expression cassettes for the constructs with the deleted (G₄S)₃ linker, with a single C1 insertion, and with four tandem C1 repeats as being representative of the entire range of CatS susceptibilities. These yeast strains displayed the expected rank order of cross-presentation efficiency: (C1)₄>C1> deleted (FIG. 4A). The gains in cross-presentation efficiency with increased CatS susceptibility were not due to the vacuolar route becoming dominant; instead, cross-presentation of all three strains remained inhibited by lactacystin and unaffected or slightly improved by chloroquine, suggesting that the antigen released by CatS moved from the phagosome to the cytosol.

### Example 5

### Evidence of a Time Window for Productive Antigen Release

With these integrated expression yeast strains, at least 98% of the yeast cells expressed the surface-displayed antigen (compared to ˜75% for transformed yeast subject to plasmid loss), so antigen loss that occurred after phagocytosis could be clearly distinguished. We developed an assay for monitoring in vivo antigen processing involving lysing the DCs at various time points after phagocytosis was initiated, followed by labeling the released yeast with α-HA and α-c-myc antibodies. Antigen release by proteolytic cleavage of the linker C-terminal to the HA epitope (or less likely, cleavage of the antigenic peptide or the c-myc epitope) results in HA⁺, c-myc⁻ yeast. We observed that between 5 and 25 min post-phagocytosis, this population was largest with the (C1)₄ linker and smallest with the deleted linker (FIG. 4B). This is consistent with CatS attacking the linkers at different rates during this early stage of phagosomal maturation, and supports the notion that early proteolytic release was responsible for the variation in cross-presentation efficiency. During the first 20 min or so, very little antigen was released in a way that would cause the loss of both epitopes (FIG. 4D), such as disulfide bond reduction or enzymatic attack of the yeast cell wall, Aga1p or Aga2p. Between 25-30 min post-phagocytosis, the double-negative population started rising rapidly, suggesting that the phagosomes had fused with late endosomes or lysosomes that provided a more acidic environment and a larger complement of active proteases. The differences in antigen loss levels between the three strains diminished at these later time points, and presumably, all the yeast cells would eventually lose their attached antigen. This suggests that antigen release rates early in phagosomal maturation are key to cross-presentation efficiency; antigen released after the 25 min time point may be mostly degraded by lysosomal proteases rather than cross-presented.

### Example 6

### Time Window of Antigen Release Exists for Maximum Cross Presentation

One of the earliest applications of yeast surface display was to perform directed evolution of a fluorescein-binding single chain variable fragment (scFv) to select for mutants with increased affinity (Boder et al., 2000). The existence of a pool of mutants spanning over four orders of magnitude in dissociation rate provided the opportunity to manipulate antigen release kinetics in a manner distinct from proteolytic release. We first loaded yeast expressing these scFv mutants with fluorescein-tagged extended peptides, but the surface display levels of these scFvs were low and variable. We then inverted the topology and loaded fluorescein-conjugated yeast with scFv-ARNLVPMVATVQGQN-c-myc fusion proteins, with the attendant advantage that the delivered antigen dose would be independent of the protein expression level. Four scFvs, with attributes listed in Table 3, were selected from the mutant pool to be produced as scFv-antigen fusions.

TABLE 3
Properties of fluorescein-binding scFvs

Dissociation half-time at

| Name | pH 7.4, 25° C.* | pH 5.4, 37° C. |
|---|---|---|
| 4M2.3 | 22 min | <1 min |
| 4M3.12 | 4.0 h | 4.8 min |
| 4M4.5 | 26 h | 9.8 min |
| 4M5.3 | 5.7 days | 1.9 h |

*From (Boder et al., 2000)

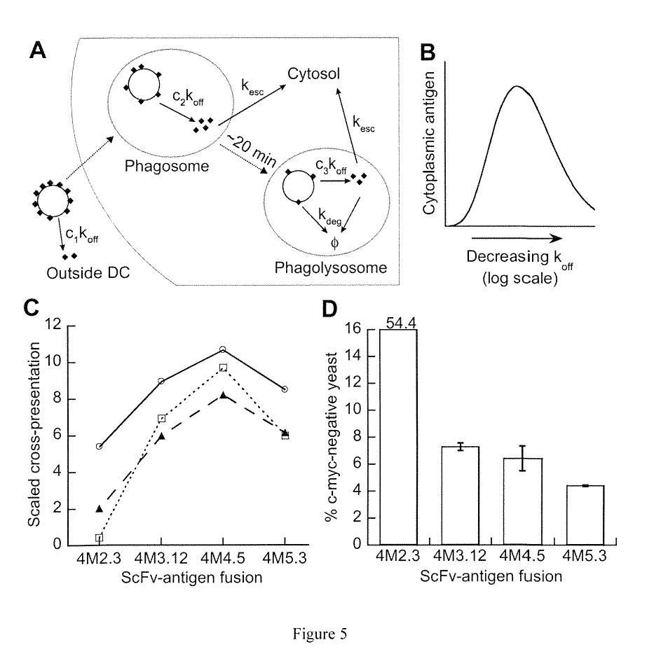

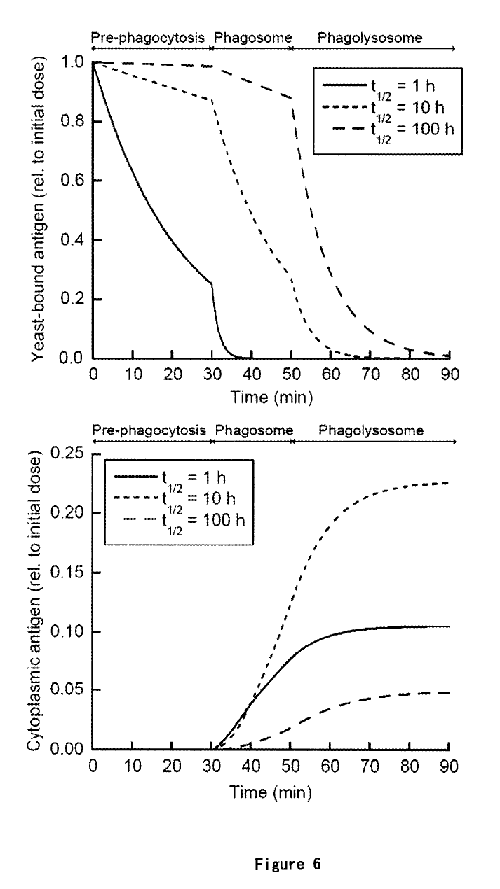

We developed a mathematical model based on the schematic in FIG. 5A to predict the cross-presentation outcome. In this model, the scFv-antigen fusion dissociates from yeast cells in three stages, the first encompassing the handling steps and time lag before phagocytosis by DCs, the second being the estimated 20 min window in phagosome maturation before the transition into a phagosolysosome, the third stage. We made the simplifying approximation that for all scFvs, the dissociation rates during these three stages could each be expressed as a proportionality constant multiplied by the measured dissociation rate at neutral pH and 25° C. (k_{off}). ScFv-antigen released prior to phagocytosis is assumed to be lost, whereas scFv-antigen released in the phagosome escapes to the cytosol at a rate k_{esc}. When the phagosome matures into a phagolysosome, proteases degrade both yeast-bound and free antigen with the rate constant k_{deg}. Protease activity that could cause antigen release rather than destruction of the epitope was neglected. We assumed that the final level of cross-presentation is proportional to the amount of antigen that escapes to the DC cytosol. The solution to the ordinary differential equations comprising this model describes a bell-shaped curve for cytosolic antigen versus k_{off} (FIG. 5B). The existence of a k_{off} value optimal for cross-presentation was a property of the model that was robust to simultaneous parameter variations spanning three orders of magnitude. At very high k_{off} values, most of the antigen is lost prior to phagocytosis, whereas at very low k_{off} values, little antigen is freed in the phagosome and the majority is degraded in the phagolysosome (see FIG. 6 for illustrative time course plots).

If antigen release rates in the phagosome did not affect cross-presentation, we would expect to see cross-presentation efficiencies rising monotonically with decreasing k_{off} due to the increased dose taken up by the DCs. Instead, the results of three independent cross-presentation experiments confirmed the existence of the model-predicted optimum, with the femtomolar fluorescein binder 4M5.3 resulting in less cross-presentation than the lower affinity 4M4.5 (FIG. 5C). When the yeast were extracted from lysed DCs 15 min post-phagocytosis, antigen loss was shown to decrease with increasing affinity (FIG. 5D). Although the dissociation half-time of 4M5.3 is almost two hours in vitro even at pH 5.4, 37° C., proteolysis by CatS or other early phagosomal proteases contributed to a baseline level of antigen release not accounted for in the model; hence, the 4M5.3-antigen fusion gave rise to higher than expected levels of cross-presentation.

Protease-accessible antigen exposed on the yeast external surface was found to be cross-presented much more efficiently than antigen trapped inside the tough cell wall; and increasing the susceptibility to CatS cleavage of the linker between the antigen and its cell wall anchor resulted in increased cross-presentation efficiency. Third, there exists an optimal affinity for antibody fragments used to attach antigen to the yeast surface, with extremely low dissociation rates being detrimental for cross-presentation efficiency.

Our analysis of antigen loss occurring post-phagocytosis suggests that there exists a limited time window for productive antigen release. In the case of yeast surface display, it appears that antigen freed after the 25 min time point did not contribute significantly to cross-presentation. The 25 min time point coincided with a sudden rise in antigen loss by means other than cleavage of the linker, possibly indicating phagosome fusion with late endosomes or lysosomes. With macrophages that had phagocytosed yeast, the phagosomal pH took 20-25 minutes to decrease to a minimum of about 5.0 (Geisow et al., 1981). The three constructs with different linkers that we compared displayed the greatest variation in antigen loss during the 10-20 min window, so it is likely that the major source of N9V peptide ultimately cross-presented was antigen freed during this time frame. With endocytosed antigen, it has been suggested that antigen destined for cross-presentation exited early from the endosomal pathway; the bulk of the antigen was colocalized with late endosomal/lysosomal markers after 25 min but did not contribute to cross-presentation (Palliser et al., 2005).

The narrow time window available for antigen release suggests that CatS, unusual among cathepsins for being active at up to neutral pHs, may play a special role in phagosome-to-cytosol cross-presentation. Roles for CatS in the vacuolar route of cross-presentation (Shen et al., 2004) and class II presentation (Pluger et al., 2002) have previously been identified.

In our mathematical model, after 20 min of phagosome maturation, proteolytic degradation of antigen competed with antigen export into the cytosol. Thus, only a small fraction of antigen released after 25 min or so contributed to cross-presentation. However, we further speculate that phagolysosome formation may in some way close off the means for antigen egress to the cytosol, thus imposing another limit on the time window for productive antigen release. Teleologically, it would make sense for the class II epitopes generated in phagolysosomes to be retained rather than exported to the cytosol. The nature of this phagosomal “pore” remains a mystery, although it appears to have a size limit, with 40 K but not 500 K dextran being translocated (Rodríguez et al., 1999). We observed that scFv-N9V attached to fluorescein-conjugated yeast, despite delivering more copies of N9V per yeast, resulted in less cross-presentation compared to surface-displayed N9V. This raises the possibility that the phagosome-to-cytosol transport mechanism is more efficient for shorter polypeptides, or favors linear/misfolded polypeptides as opposed to folded proteins. Several years ago, at least three studies provided evidence that membranes of the endoplasmic reticulum (ER) contribute to the nascent phagosome (Ackerman et al., 2003; Gagnon et al., 2002; Guermonprez et al., 2003), leading to speculation that ER-resident proteins like the Sec61 translocon or Der1p (Guermonprez and Amigorena, 2005) may be responsible. However, ER-phagosome fusion has since been convincingly disputed (Touret et al., 2005). Solving this mystery would represent a significant advance in the state-of-the-art and may permit the development of techniques to either increase the rate of antigen export to the cytosol or extend the time window for which this mechanism is active. Increasing the transport of antigen to the cytosol in this manner, combined with more complete antigen release during the critical time window, could lead to the development of more effective vaccines designed to raise cellular immunity against virus-infected cells and cancer cells.

### Example 7

### NY-ESO-1 Cancer Antigen Site-Specifically to Yeast Cell Surface is Presented to both NY-ESO-1-specific CD4⁺ and CD8⁺ T Cells

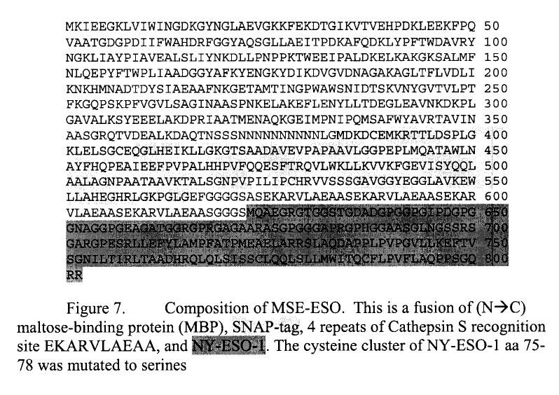

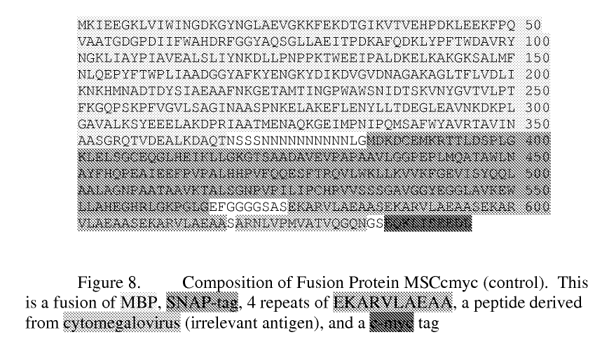

Fusion Protein Preparation Standard molecular cloning techniques were used to construct the expression plasmids starting from the pMal-c2x vector (New England Biolabs, Ipswich, Mass.). The proteins were expressed in the E. coli strain BL21(DE3)RIPL (Stratagene, La Jolla, Calif.) and purified from the bacterial lysate by affinity chromatography with amylose resin followed by size exclusion chromatography. The composition and amino acid sequence of the MSE and MSCcmyc (control) fusion proteins are shown in FIGS. 7 and 8 respectively. MBP-ESO is a fusion protein consisting only of MBP and NY-ESO-1; MBP-cmyc consists only of MBP and the c-myc tag.

Site-Specific Conjugation of the Fusion Proteins to Yeast Cell Wall MSE and MSCcmyc fusion proteins were then conjugated to the yeast strain SWH100, derived from EBY100 (Boder et al., 1997) by integrative transformation of an expression cassette for aga2p. Expression of aga1p and aga2p provides more free lysines on the yeast cell wall, increasing protein conjugation by 3-6 fold. Induced SWH100 yeast (5 OD.ml) was washed thoroughly with PBS, UV-irradiated, and resuspended in 50 μl dimethylformamide containing 1 mg BG-GLA-NHS (amine-reactive benzyl guanine, Covalys Biosciences AG, Witterswil, Switzerland). After 2 h incubation at 30° C., the BG-derivatized yeast was washed thoroughly with PBS+0.1% BSA. The yeast pellet was resuspended in 2.5 ml PBS containing 12 μM of either MSE or MSCcmyc and incubated at 30° C. for 4 h, allowing the SNAP-tag to react with the BG moieties. The yeast was washed with PBS+0.1% BSA before use.

Non-Site-Specific Chemical Conjugation of Fusion Proteins to Yeast Cell Wall BJ5α yeast (5 OD.ml) was washed thoroughly with pH 5.5 0.1 M sodium acetate buffer, UV-irradiated and incubated with 10 mM sodium meta-periodate in the same buffer for 20 min on ice, thus forming aldehyde groups on the yeast cell wall. The yeast was washed and incubated with 0.25 ml of either MBP-ESO or MBP-cmyc (5 mg/ml in PBS) and 2.5 μl of 1 M sodium cyanoborohydride for 4 days at 4° C. Unreacted aldehydes were then quenched by reaction with 12.5 μl of Tris-HCl for 30 min at room temperature. The yeast was washed with PBS+0.1% BSA before use.

Assays for Antigen Presentation. To test for antigen presentation, MSE or MSCcmyc yeasts were added to monocyte-derived dendritic cells (DCs) at a 20:1 ratio. Sixteen to twenty hours later, the DCs were washed and co-cultured for 24 hours with NY-ESO-1-specific CD8⁺ or CD4⁺ T cell clone that is restricted to HLA-Cw3 or HLA-DP04, respectively.

An ELISPOT assay was employed to quantify the number of interferon gamma (IFN-γ) secreting cells as a result of recognition of NY-ESO-1 peptide presented by DCs. Fifty thousand of DCs and the indicated number of T cell clone were added on the wells of an ELISPOT plate.

T Cell Clones. Generation of HLA-Cw3-restricted is previously described (Nagata et al., Proc Natl Acad Sci USA. 2002, 99:10629-10634). HLA-Cw3-restricted, NY-ESO-1-specific CD8⁺ T cell clone was generated from a melanoma patient NW29 (Gnjatic et al., 2000, Proc. Natl. Acad. Sci. USA 97: 10917-10922). The CD4⁺ T helper clone recognizing NY-ESO-1 peptide (157-170) on HLA-DP4 was established by stimulation of PBMC of an ovarian cancer patient with NY-ESO-1157-170 peptide followed by a limiting dilution.

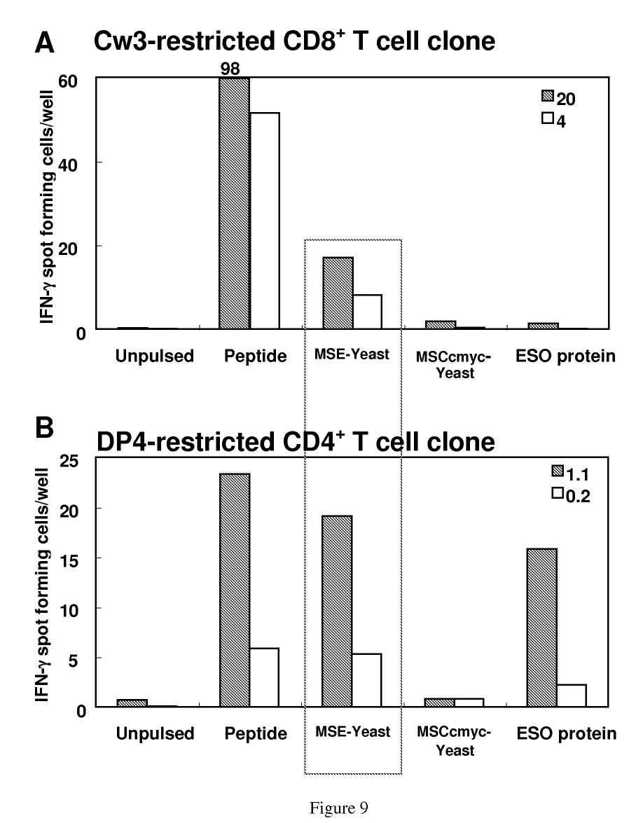

Results: FIG. 9A shows that the NY-ESO-1-derived epitope (peptide 92-100) in MSE-yeast was loaded on HLA-Cw3 to stimulate an HLA-Cw3-restricted CD8⁺ T cell clone at levels far exceeding the background caused by the control MSCcmyc-yeast lacking NY-ESO-1. FIG. 9B shows that in addition to causing cross-presentation, MSE-yeast was processed by DCs to stimulate a DP4-restricted CD4⁺ T helper cell clone recognizing NY-ESO-1 peptide 157-170. Such T cell help is important in enhancing cellular and humoral responses to vaccines in vivo.

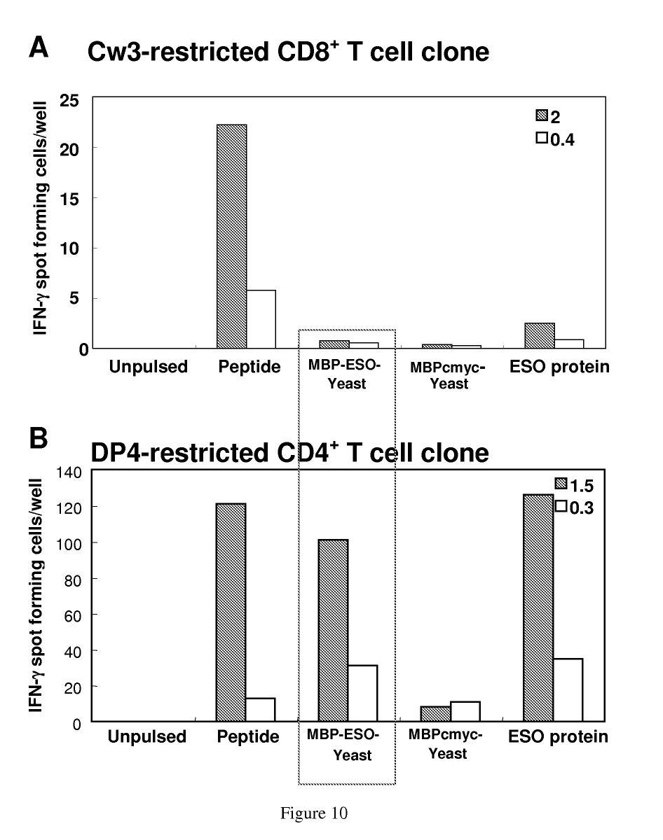

In comparison, and as shown in FIG. 10A, the HLA-Cw3-restricted CD8⁺ T cell clone was not able to recognize DCs pulsed with yeast chemically conjugated by reductive amination to MBP-ESO. This indicates that cross-presentation was contingent upon site-specific conjugation in a configuration that facilitated antigen release in the phagosome. With MSE-yeast, the only covalent bond between the fusion protein and the yeast was through the SNAP-tag domain, which was separated from the NY-ESO-1 domain by Cathepsin S cleavage sites. With MBP-yeast, lysine residues within the NY-ESO-1 domain were covalently linked to the yeast cell wall, inhibiting release in the early phagosome. MHC class II presentation to the HLA-DP4-restricted CD4⁺ T cells, however, was still efficient with chemically conjugated antigen, as shown in FIG. 10B.

### Example 8

### Experimental Procedures

Cells: Human HLA-A*0201 monocytes were obtained from two sources, purified either by counter-flow centrifugal elutriation (Advanced Biotechnologies Inc, Columbia, Md.) or negative magnetic cell sorting (Biological Specialty Corporation, Colmar, Pa.). Similar results were obtained with both sources. The monocytes were aliquoted into vials and cryopreserved in 90% fetal bovine serum (FBS), 10% DMSO. For each experiment, one or more vials were thawed and washed in C10 medium: RPMI 1640 with 10% FBS, 2 mM L-glutamine, 10 mM HEPES, 1 mM sodium pyruvate, 1× non-essential amino acids, 50 μM β-mercaptoethanol and Primocin (InvivoGen, San Diego, Calif.). Unless otherwise indicated, media components were from Hyclone (Logan, Utah); low endotoxin products were chosen where available. 4-5×10⁶ monocytes were cultured per well of a 6-well plate in 2.5 ml C10 medium supplemented with 1000 U/ml each of interleukin-4 and granulocyte-macrophage colony stimulating factor (C10GF; cytokines from R & D Systems, Minneapolis, Minn.). After 2 and 4 days of culture, each well was topped up with 0.5 ml C10GF; after 6 days of culture, floating and loosely adherent monocyte-derived DCs were harvested by gentle resuspension.

Vials of a human CD8⁺ T cell line specifically recognizing the peptide NLVPMVATV in the context of HLA-A*0201 were purchased from ProImmune (Oxford, UK). Each vial was thawed and cultured overnight in RPMI 1640 with 10% FBS and 50 ng/ml interleukin-2 and used the next day.

Yeast Surface Display Plasmids for yeast surface display were based on pCT-CON (Colby et al., 2004) and were transformed into EBY100 (Boder and Wittrup, 1997), a strain that expresses Aga1p under galactose induction, using the Frozen EZ Yeast Transformation II Kit (Zymo Research, Orange, Calif.). The Supplementary Data below provides details on plasmid construction. Yeast colonies were cultured to mid-log phase at 30° C. in selective SD-CAA medium (2% dextrose, 0.67% yeast nitrogen base, 0.5% casamino acids, 0.1 M sodium phosphate, pH 6.0) and then induced in SG-CAA (SD-CAA with galactose replacing dextrose) for 48 h at 20° C. Single copies of some expression cassettes were integrated into the EBY100 yeast chromosome using the integrating shuttle vector pRS304 (Sikorski and Hieter, 1989). The resulting yeast strains were grown up in rich YPD medium (1% yeast extract, 2% peptone, 2% dextrose) and induced in YPG (1% yeast extract, 2% peptone, 2% galactose) for 36 h at 20° C. Yeast media nitrogen sources were obtained from BD (Franklin Lakes, N.J.). Surface display levels were measured by flow cytometry with chicken a-c-myc (Invitrogen, Carlsbad, Calif.) or 9e10 monoclonal antibody (Covance, Princeton, N.J.). The number of copies per yeast cell was estimated by comparison with Quantum Simply Cellular beads (Bangs Labs, Fishers Ind.).

Cross-presentation Assay After 6 days of differentiation, monocyte-derived DCs were seeded in 96-well round bottom plates at 1 or 2×10⁵ cells in 200 μl C10GF per well. Appropriate numbers of yeast cells (measured by optical density at 600 nm with 10D≈10⁷/ml) were rendered non-viable by UV-irradiation (2×1000 J/m² in a Stratalinker, Stratagene, La Jolla, Calif.), pelleted by centrifugation and added to the DCs. For inhibition experiments, DCs were pre-incubated with lactacystin (5 μM; Calbiochem, San Diego, Calif.) or chloroquine (25 μM) for one hour before yeast samples were introduced. 24 h later, half the medium was replaced with a T cell suspension, with 0.7-1×10⁵ T cells per well. Following 4 h of co-culture, the contents of each well were transferred to tubes for labeling with Miltenyi's IFN_{Y} secretion assay kit (Bergisch Gladbach, Germany) according to the recommended protocol. Briefly, cells were labeled with a bispecific antibody that captures secreted IFN_{Y} on the cell surface during a 45 min incubation period in medium at 37° C., and then labeled on ice for 30 min with α-CD8-FITC (BD) and α-IFN_{Y}-PE (Miltenyi). In experiments involving FITC-conjugated yeast, Q-CD8-Alexa Fluor 647 (BD) was substituted. The percentage of CD8⁺ cells that were IFN_{Y}_{⁺} was determined by flow cytometry (Coulter Epics XL, Fullerton, Calif. or BD FACSCalibur). The cut-off PE fluorescence was set for each experiment such that about 0.5% of T cells were IFN_{Y}_{⁺} in a negative control sample (no yeast or peptide). The positive control with 1 μM of the extended peptide ARNLVPMVATVQGQN (synthesized by GenScript, Piscataway, N.J.) resulted in 45-70% IFN_{Y}_{⁺} T cells.

Yeast Intracellular Expression Intracellular expression of the same fusion protein as is expressed by surface display was achieved by deleting the signal peptide of Aga2p, followed by transformation into BJ5464α (Yeast Genetic Stock Center, Berkeley, Calif.). BJ5464α is isogenic to the parent strain of EBY100 and lacks the galactose-inducible Aga1p gene. The resulting colonies were grown up in SD-CAA and induced in SG-CAA for 12 h at 30° C.

Slot Blot Comparison of Antigen Levels 6 OD.ml of each yeast culture was washed with phosphate-buffered saline (PBS), resuspended in 300 μl 25 mM Tris(2-carboxyethyl)phosphine hydrochloride (TCEP, Soltec Ventures, Beverly, Mass.) in PBS, and incubated for on ice for 30 min. The proteins released into solution by the reducing agent were pooled with those from a second 30 min extraction with 25 mM TCEP. The yeast pellets were then washed with spheroplast buffer (50 mM Tris-HCl, pH 7.5, 1.4 M sorbitol, 40 mM β-mercaptoethanol), incubated with 2.4 U Zymolyase (Zymo Research) in 120 μl spheroplast buffer containing a protease inhibitor cocktail (Roche, Indianapolis, Ind.) for 15 min at 37° C., and boiled in 2% sodium dodecyl for 5 min. The protein extracts were blotted onto nitrocellulose membrane with a slot-blotting apparatus (Bio-rad, Hercules, Calif.). The membrane was blocked with 5% milk powder, incubated with 9e10 ascites fluid (Covance) followed by goat α-mouse-horse radish peroxidase (Pierce, Rockford, Ill.), developed with SuperSignal West Dura substrate (Pierce), and imaged on a Fluor S Imager (Bio-rad).

Surface Display Antigen Dose Normalization In experiments where different linkers were used to surface-display antigen, several cultures of each yeast sample were induced, and cultures with mean antigen levels within 10% of each other were selected to minimize the effect of variable antigen dose on cross-presentation. However, the variability in expression level across the panel of initial constructs (deleted linker, unchanged, and C₁₋₅) was too high for this approach to be satisfactory. Therefore, each yeast sample was mixed with the appropriate amount of EBY100 yeast to normalize the antigen dose while maintaining the 20:1 ratio of yeast to DCs.

Measuring Linker Susceptibility to CatS

0.2 OD.ml of each yeast sample was washed and incubated with the indicated amounts of recombinant human CatS (Calbiochem) in 100 μl PBS at 37° C. The yeast samples were washed and labeled with 12CA5 monoclonal antibody (α-HA; Roche) and chicken α-c-myc, followed by goat α-mouse-PE (Sigma-Aldrich, St. Louis, Mo.) and goat α-chicken-Alexa Fluor 488 (Invitrogen). The mean c-myc fluorescence of the HA⁺ population was compared against that of yeast samples that had not been treated with CatS.

Post-phagocytosis Analysis DCs (2×10⁵/well) were seeded in 96-well round bottom plates, with separate plates for each time point. After adding the yeast samples (5×10⁵/well), the plates were immediately centrifuged briefly (200×g, 1 min) to settle the yeast and were returned to the incubator. At each time point, a plate was placed on ice and 90% of the medium in each well was replaced with cold RIPA buffer (Sigma-Aldrich). The well contents were moved to tubes, vortexed to promote cell lysis, and centrifuged to pellet the released yeast. The yeast was washed with RIPA buffer and PBS with 0.1% bovine serum albumin (BSA) before being labeled for HA and c-myc epitopes as described above.

Fluorescein-binding ScFvs The fluorescein-binding scFvs used here were products of directed evolution for decreased dissociation rate using yeast surface display (Boder et al., 2000). These scFvs were subcloned into pRS316-based plasmids with an improved alpha mating factor pre-pro sequence (Rakestraw et al., unpublished). Codons encoding the extended peptide ARNLVPMVATVQGQN were inserted between the scFv C-terminus and the c-myc epitope. The resulting constructs were transformed into the protein disulfide isomerase-overexpressing yeast strain YVH10 (Robinson et al., 1994) together with a dummy plasmid bearing the trp nutritional marker. Transformants were grown up in SD-CAA and induced in YPG containing 0.1 M sodium phosphate, pH 6.0 for 3 days at 20° C. The culture supernatants containing approximately 10 mg/L of scFv-antigen were adjusted to pH 7.4 and dialyzed against PBS.

Fluorescein-conjugated Yeast UV-irradiated BJ5654a yeast cells were washed three times in 0.4 M sodium carbonate, pH 8.4 and resuspended in 10 μl/OD.ml of a freshly prepared 1.5 mg/μl solution of fluorescein-PEG-NHS (MW 5000; Nektar, Huntsville, Ala.) in sodium carbonate buffer. The reaction was allowed to proceed for 30 min at room temperature, after which the yeast was washed six times with PBS containing 0.1% BSA. Fluorescein-conjugated yeast was loaded with antigen by incubation with scFv-antigen culture supernatants (1 ml per 107 yeast) for 1 hour on ice. Flow cytometry analysis (c-myc labeling) of the loaded yeast showed that the antigen levels mediated by 4M2.3, 4M3.12 and 4M4.5 were within ˜5% of each other, but the level of 4M5.3-antigen was about 15% higher. Labeling fluorescein-conjugated yeast with 4M5.3 fusion protein for 30 min followed by 30 min, 37° C. incubation in pH 5.4 PBS containing 0.1% BSA and 1 μM fluorescein-biotin resulted in a final antigen level comparable to that mediated by the other scFvs. This method of antigen level normalization was performed for the cross-presentation assay. In addition, to reduce antigen loss before phagocytosis, the plate was centrifuged (200×g, 1 min) immediately after addition of the yeast to the DCs.

NY-ESO-1-specific T cells clones The following T cell clones were used in experiments. C6: HLA-Cw3-restricted CD8⁺ T cell clone which recognize NY-ESO-1 92-100 peptide; and VK/D7F6: HLA-DP4-restricted CD4⁺ T cell clone which recognizes NY-ESO-1 157-170 peptide.

Preparation of Monocyte-Derived Dendritic Cells (Mo-DC). Human monocytes were isolated from peripheral blood mononuclear cells (PBMC) of healthy donors by magnetic sorting using CD14 beads (Miltenyi Biotec). Monocytes were cultured in 6 well plates in the presence of 20 ng/ml GM-CSF and 20 ng/ml IL-4 (both from R&D systems) for 6 days to differentiate into Mo-DC. The culture medium used for the generation of Mo-DC was RPMI medium supplemented with 2.5% FCS, penicillin, streptomycin, L-Glutamine. On day 6, non-adherent Mo-DC were harvested by pipetting and pulsed overnight with or NY-ESO-1 protein, peptide, or Yeast construct.

ELISPOT Assay Antigen-pulsed Mo-DC (typically 50,000 cells/well) and NY-ESO-1-specific CTL or indicated number of helper T cell clone were washed twice and resuspended in RPMI medium. They were seeded to anti-IFN-γ mAb (1-DIK, Mabtech)-precoated mixed cellulose ester membrane filter plate (Millipore) and incubated for 24 hours in 5% CO2 37° C. incubator. The plate was developed with biotinylated anti-IFN-γ mAb (7-B6-1, Mabtech), Streptavidin-AP conjugate (Roche) and BCIP/NBT alkaline Phosphatase Substrate (Sigma). The number of spots was evaluated on CTL Immunospots analyzer.

Supplementary Data

Construction of pCT-N9V pCT-N9V is the plasmid bearing the surface display construct that was integrated into the genome of EBY100 to create EBYN9V. The oligonucleotides

(SEQ ID NO:6)

5′-TAGCGCTAGAAATTTGGTTCCAATGGTTGCTACTGTTCAAGGTCAAA
ACG
and

(SEQ ID NO:7)

5′-ATCCGTTTTGACCTTGAACAGTAGCAACCATTGGAACCAAATTTCTA
GCG

were annealed to form a double-stranded fragment encoding the peptide ARNLVPMVATVQGQN (SEQ ID NO: 5) flanked by NheI and BamHI-compatible overhangs. This fragment was ligated with pCT-CON vector digested with NheI and BamHI.

Construction of pCTc-N9V The aga2p sequence in pCT-N9V was cut out with EcoRI and PstI and replaced with a PCR product of the same gene minus the signal peptide, amplified with primers containing the same restriction sites. This construct was transformed into BJ5464α yeast to allow intracellular expression of the aga2p-antigen fusion protein.

Insertion of C1 to C5 sequences Codons encoding the following sequences were inserted at the NheI site of pCT-N9V by annealing pairs of oligonucleotides such that NheI-compatible overhangs were produced at either end. Upon ligation into pCT-N9V, the NheI site was regenerated at the C-terminal end of the insert but destroyed at the N-terminal end. This allowed the procedure to be repeated to insert tandem copies of each insert. Constructs were sequenced to ensure that the inserts were in the correct orientation.

List of Linker Sequences and Oligonucleotides

List of linker sequences and oligonucleotides

C1: EKARVLAEA (SEQ ID NO:8)

| | |
|---|---|
| 5′-CTAGTGAAAAAGCTAGAGTTTTGGCTGAAGCTG | (SEQ ID NO:9) |
| 5′-CTAGCAGCTTCAGCCAAAACTCTAGCTTTTTCA | (SEQ ID NO:10) |
| C2: SSAESLK (SEQ ID NO:11) | |
| 5′-CTAGTTCTTCTGCTGAATCTTTGAAAG | (SEQ ID NO:12) |
| 5′-CTAGCTTTCAAAGATTCAGCAGAAGAA | (SEQ ID NO:13) |
| C3: NWVCAAKF (SEQ ID NO:14) | |
| 5′-CTAGTAATTGGGTTTGTGCTGCTAAATTTG | (SEQ ID NO:15) |
| 5′-CTAGCAAATTTAGCAGCACAAACCCAATTA | (SEQ ID NO:16) |
| C4: GILQINSR (SEQ ID NO:17) | |
| 5′-CTAGTGGTATTTTGCAGATTAATTCTAGAG | (SEQ ID NO:18) |
| 5′-CTAGCTCTAGAATTAATCTGCAAAATACCA | (SEQ ID NO:19) |
| C5: QWLGAPVP (SEQ ID NO:20) | |
| 5′-CTAGTCAATGGTTGGGTGCTCCAGTTCCAG | (SEQ ID NO:21) |
| 5′-CTAGCTGGAACTGGAGCACCCAACCATTGA | (SEQ ID NO:22) |

Deletion of the (G4S)₃ linker pCT-N9V was digested with PstI and NheI to remove the codons encoding the (G4S)₃ linker. The Klenow fragment of DNA Polymerase I was used to remove the 3′ overhang generated by PstI and fill in the 5′ extension generated by NheI, permitting the blunt ends to be ligated in-frame.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. Furthermore, the teachings and disclosures of all references cited herein are expressly incorporated in their entireties by reference.

### REFERENCES

Accapezzato, D., Visco, V., Francavilla, V., Molette, C., Donato, T., Paroli, M., Mondelli, M. U., Doria, M., Torrisi, M. R., and Barnaba, V. (2005). Chloroquine enhances human CD8+ T cell responses against soluble antigens in vivo. J Exp Med 202, 817-828.
Ackerman, A. L., Kyritsis, C., Tampe, R., and Cresswell, P. (2003). Early phagosomes in dendritic cells form a cellular compartment sufficient for cross presentation of exogenous antigens. PNAS 100, 12889-12894.
Bachmann, M. F., Oxenius, A., Pircher, H., Hengartner, H., Ashton-Richardt, P. A., Tonegawa, S., and Zinkernagel, R. M. (1995). TAP1-independent loading of class I molecules by exogenous viral proteins. Eur J Immunol 25, 1739-1743.
Barron, M. A., Blyveis, N., Pan, S. C., and Wilson, C. C. (2006). Human Dendritic Cell Interactions with Whole Recombinant Yeast: Implications for HIV-1 Vaccine Development. J Clin Immunol 26, 251-264.
Baumgrass, R., Williamson, M. K., and Price, P. A. (1997). Identification of peptide fragments generated by digestion of bovine and human osteocalcin with the lysosomal proteinases cathepsin B, D, L, H, and S. J Bone Miner Res 12, 447-455.
Boder, E. T., Midelfort, K. S., and Wittrup, K. D. (2000). Directed evolution of antibody fragments with monovalent femtomolar antigen-binding affinity. Proc Natl Acad Sci USA 97, 10701-10705.
Boder, E. T., and Wittrup, K. D. (1997). Yeast surface display for screening combinatorial polypeptide libraries. Nat Biotechnol 15, 553-557.
Breinig, F., Heintel, T., Schumacher, A., Meyerhans, A., and Schmitt, M. J. (2003). Specific activation of CMV-primed human T lymphocytes by cytomegalovirus pp 65 expressed in fission yeast. FEMS Immunology and Medical Microbiology 38, 231-239.
Brinkman, J. A., Fausch, S. C., Weber, J. S., and Kast, W. M. (2004). Peptide-based vaccines for cancer immunotherapy. Expert Opinion on Biological Therapy 4, 181-198.
Colby, D. W., Kellogg, B. A., Graff, C. P., Yeung, Y. A., Swers, J. S., and Wittrup, K. D. (2004). Engineering Antibody Affinity by Yeast Surface Display Methods in Enzymology. In Protein Engineering, D. E. R. a. J. P. Noel, ed. (Academic Press), pp. 348-358.
Devin B. Lowe, M. H. S., Ronald C. Kennedy, (2006). DNA vaccines: Successes and limitations in cancer and infectious disease. Journal of Cellular Biochemistry 98, 235-242.
Gagnon, E., Duclos, S., Rondeau, C., Chevet, E., Cameron, P. H., Steele-Mortimer, O., Paiement, J., Bergeron, J. J., and Desjardins, M. (2002). Endoplasmic reticulum-mediated phagocytosis is a mechanism of entry into macrophages. Cell 110, 119-131.
Gattinoni, L., Powell, D. J., Rosenberg, S. A., and Restifo, N. P. (2006). Adoptive immunotherapy for cancer: building on success. 6, 383-393.
Geisow, M., D'Arcy Hart, P., and Young, M. (1981). Temporal changes of lysosome and phagosome pH during phagolysosome formation in macrophages: studies by fluorescence spectroscopy. J Cell Biol 89, 645-652.
Guermonprez, P., and Amigorena, S. (2005). Pathways for antigen cross presentation. Springer Semin Immunopathol 26, 257-271.
Guermonprez, P., Saveanu, L., Kleijmeer, M., Davoust, J., Van Endert, P., and Amigorena, S. (2003). ER-phagosome fusion defines an MHC class I cross-presentation compartment in dendritic cells. Nature 425, 397-402.
Koelle, D. M. (2006). Vaccines for herpes simplex virus infections. Curr Opin Investig Drugs 7, 136-141.
Kovacsovics-Bankowski, M., Clark, K., Benacerraf, B., and Rock, K. (1993). Efficient Major Histocompatibility Complex Class I Presentation of Exogenous Antigen Upon Phagocytosis by Macrophages. PNAS 90, 4942-4946.
Kovacsovics-Bankowski, M., and Rock, K. L. (1995). A phagosome-to-cytosol pathway for exogenous antigens presented on MHC class 1 molecules. Science 267, 243-246.
Lennon-Dumenil, A.-M., Bakker, A. H., Maehr, R., Fiebiger, E., Overkleeft, H. S., Rosemblatt, M., Ploegh, H. L., and Lagaudriere-Gesbert, C. (2002). Analysis of Protease Activity in Live Antigen-presenting Cells Shows Regulation of the Phagosomal Proteolytic Contents During Dendritic Cell Activation. J Exp Med 196, 529-540.
Lollini, P.-L., Cavallo, F., Nanni, P., and Formi, G. (2006). Vaccines for tumour prevention. 6, 204-216.
Lu, Y., Bellgrau, D., Dwyer-Nield, L. D., Malkinson, A. M., Duke, R. C., Rodell, T. C., and Franzusoff, A. (2004). Mutation-selective tumor remission with Ras-targeted, whole yeast-based immunotherapy. Cancer Res 64, 5084-5088.
McMichael, A. J. (2006). HIV VACClNES. Annual Review of Immunology 24, 227-255.
Morse, M. A., Chui, S., Hobeika, A., Lyerly, H. K., and Clay, T. (2005). Recent developments in therapeutic cancer vaccines. Nat Clin Pract Oncol 2, 108-113.
Palliser, D., Guillen, E., Ju, M., and Eisen, H. N. (2005). Multiple Intracellular Routes in the Cross-Presentation of a Soluble Protein by Murine Dendritic Cells. J Immunol 174, 1879-1887.
Pfeifer, J. D., Wick, M. J., Roberts, R. L., Findlay, K., Normark, S. J., and Harding, C. V. (1993). Phagocytic processing of bacterial antigens for class I MHC presentation to T cells. 361, 359-362.
Pillay, C. S., Elliott, E., and Dennison, C. (2002). Endolysosomal proteolysis and its regulation. Biochem J 363, 417-429.
Pluger, E. B., Boes, M., Alfonso, C., Schroter, C. J., Kalbacher, H., Ploegh, H. L., and Driessen, C. (2002). Specific role for cathepsin S in the generation of antigenic peptides in vivo. Eur J Immunol 32, 467-476.
Robinson, A. S., Hines, V., and Wittrup, K. D. (1994). Protein disulfide isomerase overexpression increases secretion of foreign proteins in Saccharomyces cerevisiae. Biotechnology (N Y) 12, 381-384.
Rock, K. L., and Shen, L. (2005). Cross-presentation: underlying mechanisms and role in immune surveillance. Immunological Reviews 207, 166-183.
Rodriguez, A., Regnault, A., Kleijmeer, M., Ricciardi-Castagnoli, P., and Amigorena, S. (1999). Selective transport of internalized antigens to the cytosol for MHC class I presentation in dendritic cells. Nat Cell Biol 1, 362-368.
Shen, L., Sigal, L. J., Boes, M., and Rock, K. L. (2004). Important Role of Cathepsin S in Generating Peptides for TAP-Independent MHC Class I Crosspresentation In Vivo. Immunity 21, 155-165.
Shiina, M., and Rehermann, B. (2006). Hepatitis C vaccines: Inducing and challenging memory T cells. Hepatology 43, 1395-1398.
Sikorski, R. S., and Hieter, P. (1989). A System of Shuttle Vectors and Yeast Host Strains Designed for Efficient Manipulation of DNA in Saccharomyces cerevisiae. Genetics 122, 19-27.
Srivastava, P. K. (2006). Therapeutic cancer vaccines. Current Opinion in Immunology Lymphocyte development/Tumour immunology 18, 201-205.
Stubbs, A. C., Martin, K. S., Coeshott, C., Skaates, S. V., Kuritzkes, D. R., Bellgrau, D., Franzusoff, A., Duke, R. C., and Wilson, C. C. (2001). Whole recombinant yeast vaccine activates dendritic cells and elicits protective cell-mediated immunity. Nat Med 7, 625-629.
Thurmond, R. L., Sun, S., Sehon, C. A., Baker, S. M., Cai, H., Gu, Y., Jiang, W., Riley, J. P., Williams, K. N., Edwards, J. P., and Karlsson, L. (2004). Identification of a Potent and Selective Noncovalent Cathepsin S Inhibitor. J Pharmacol Exp Ther 308, 268-276.
Touret, N., Paroutis, P., Terebiznik, M., Harrison, R. E., Trombetta, S., Pypaert, M., Chow, A., Jiang, A., Shaw, J., and Yip, C. (2005). Quantitative and Dynamic Assessment of the Contribution of the ER to Phagosome Formation. Cell 123, 157-170.
Trivedi, D., Williams, R. Y., O'Reilly, R. J., and Koehne, G. (2005). Generation of CMV-specific T lymphocytes using protein-spanning pools of pp 65-derived overlapping pentadecapeptides for adoptive immunotherapy. Blood 105, 2793-2801.
Underhill, D. M. (2003). Macrophage recognition of zymosan particles. J Endotoxin Res 9, 176-180.
Zaliauskiene, L., Kang, S., Sparks, K., Zinn, K. R., Schwiebert, L. M., Weaver, C. T., and Collawn, J. F. (2002). Enhancement of MHC Class II-Restricted Responses by Receptor-Mediated Uptake of Peptide Antigens. J Immunol 169, 2337-2345.

What is claimed is:

1. A method for eliciting in an animal in need thereof a cell-mediated immune response to an antigen, the method comprising
(1) providing an antigen preparation comprising a particle having a surface on which the antigen is attached, wherein upon phagocytosis of the particle by an antigen presenting cell, at least a proportion of the antigen is released from the particle in a phagosome before the phagosome fuses with a late endosome or a lysosome, and wherein at least a portion of the antigen is cross-presented on a Class I MHC molecule, and
(2) administering the antigen preparation to the animal.

2. The method of claim 1, wherein the antigen released from the particle has a molecular weight of less than about 500 kDa.

3. The method of claim 1, wherein the antigen is a protein or a derivative thereof.

4. The method according to claim 1, wherein the antigen is a cancer antigen.

5. The method according to claim 4, wherein the cancer antigen is selected from the group consisting of New York Esophageal 1 antigen (NY-ESO-1), MAGE-A1, MAGE-A2, MAGE-A3, MAGE-A4, MAGE-A6, MAGE-A8, MAGE-A10, MAGE-B, MAGE-C1, MAGE-C2, L antigen (LAGE), synovial sarcoma X breakpoint 2 (SSX2), SSX4, SSX5, preferentially expressed antigen of melanoma (PRAME), Melan-A, Tyrosinase, MAGF, PSA, CEA, HER2/nev, MART1, BCR-abl; and a mutant oncogenic form of p53, ras, myc or RB-1.

6. The method of claim 1, wherein the particle has a size that allows effective phagocytosis by the APC.

7. The method of claim 4, wherein the particle has a diameter or a cross section that ranges between about 0.3 μm and about 20 μm.

8. The method according to claim 1, wherein the antigen presenting cell is a dendritic cell.

9. The method according to claim 3, wherein the particle is a genetically engineered host cell transformed with an expression vector, and wherein the antigen is a fusion protein encoded by the expression vector, and wherein the fusion protein comprises (1) an antigenic peptide, (2) a surface anchor sequence for anchoring the fusion protein to the surface of the host cell, and (3) a protease recognition site that lies between the antigenic peptide and the surface anchor sequence, wherein the protease recognition site is recognized by a protease in the phagosome to release the antigenic peptide the host cell surface rapidly inside the phagosome.

10. The method according to claim 9, wherein the host cell is a yeast cell.

11. The method according to claim 10, wherein the yeast is Saccharomyces cerevisiae.

12. The method according to claim 10, wherein the yeast is strain EBY100.

13. The method according to claim 10, wherein the surface anchor sequence is a yeast mating adhesion receptor subunit Aga2p.

14. The method according to claim 13, wherein the antigen is linked to the signal sequence via at least a G₄S linker.

15. The method according to claim 14, wherein the protease recognition site is a Cathepsin S (CatS) recognition site.

16. The method according to claim 3, wherein the particle is a cell on whose wall a fusion protein comprising the antigen is attached via conjugation.

17. The method according to claim 16, wherein the antigen is attached via chemical conjugation or protein-mediated site-specific conjugation.

18. The method according to claim 16, wherein the host cell is a yeast cell.

19. The method according to claim 17, wherein the yeast is Saccharomyces cerevisiae.

20. The method according to claim 18, wherein the surface anchor sequence is a yeast mating adhesion receptor subunit Aga2p.

21. The method according to claim 20, wherein the antigen is linked to the signal sequence via at least a G₄S linker.

22. The method according to claim 21, wherein the protease recognition site is a Cathepsin S (CatS) recognition site.

23. The method according to claim 19, wherein the yeast is strain SWH100.

24. The method according to claim 23, wherein the host cell is non-viable.

25. The method according to claim 16, wherein the fusion protein comprises a fusion of a maltose-binding protein, SNAP-tag, 4 repeats of Cathepsin S recognition site EKARVLAEAA, and NY-ESO-1 as the antigen.

26. The method according to claim 25, wherein the fusion protein comprises an amino acid sequence of SEQ ID NO:1.

27. The method according to claim 1, wherein the particle is a pharmaceutically acceptable preparation of fungal or bacterial cell wall.

28. The method according to claim 27, wherein the particle is zymosan or a yeast cell wall preparation.

29. The method according to claim 1, wherein the particle comprises polymer beads, inorganic particles, micelles or colloidal complexes.

30. The method according to claim 29, wherein the polymer beads comprises latex beads, poly(lactic-co-glycolic acid) beads, polystyrene beads, or chitosan beads.

31. The method according to claim 29, wherein the inorganic particles are selected from the group consisting of iron oxide particles, glass beads, silica beads, gold particles, and Quantum Dots™.

32. The method according to claim 29, wherein the particles comprise Immune-stimulating complexes (ISCOMs).

33. The method according to claim 29, wherein the particles comprises liposomes.

34. A composition comprising a particle having a surface on which an isolated antigen is attached, wherein the antigen is releasable from the particle in a phagosome upon phagocytosis of the particle by an antigen presenting cell before the phagosome fuses with a late endosome or a lysosome, and wherein at least a portion of the antigen is cross-presented on a Class I MHC molecule.

35. The composition of claim 34, wherein the antigen released from the particle has a molecular weight of less than about 500 kDa

36. The composition of claim 34, wherein the antigen is a protein or a derivative thereof.

37. The composition of claim 34, wherein the antigen is a cancer antigen.

38. The composition of claim 37, wherein the cancer antigen is selected from the group consisting of New York Esophageal 1 antigen (NY-ESO-1), MAGE-A1, MAGE-A2, MAGE-A3, MAGE-A4, MAGE-A6, MAGE-A8, MAGE-A10, MAGE-B, MAGE-C1, MAGE-C2, L antigen (LAGE), synovial sarcoma X breakpoint 2 (SSX2), SSX4, SSX5, preferentially expressed antigen of melanoma (PRAME), Melan-A, Tyrosinase, MAGF, PSA, CEA, HER2/nev, MART1, BCR-abl; and a mutant oncogenic form of p53, ras, myc or RB-1.

39. The composition of claim 34, wherein the particle has a size that allows effective phagocytosis by the APC.

40. The composition of claim 39, wherein the particle has a diameter or a cross section that ranges between about 0.3 μm and about 20 μm.

41. The composition of claim 34, wherein the antigen presenting cell is a dendritic cell.

42. The composition of claim 36, wherein the particle is a genetically engineered host cell transformed with an expression vector, and wherein the antigen is a fusion protein encoded by the expression vector, and wherein the fusion protein comprises (1) an antigenic peptide, (2) a surface anchor sequence for anchoring the fusion protein to the surface of the host cell, and (3) a protease recognition site that links the antigenic peptide with the surface anchor sequence, wherein the protease recognition site is recognized by a protease in the phagosome to release the antigenic peptide the host cell surface rapidly inside the phagosome.

43. The composition of claim 42, wherein the host cell is a yeast cell.

44. The composition of claim 43, wherein the yeast is Saccharomyces cerevisiae.

45. The composition of claim 44, wherein the yeast is strain EBY100.

46. The composition of claim 43, wherein the surface anchor sequence is a yeast mating adhesion receptor subunit Aga2p.

47. The composition of claim 46, wherein the antigen is linked to the signal sequence via at least a G₄S linker.

48. The composition of claim 47, wherein the protease recognition site is a Cathepsin S (CatS) recognition site.

49. The composition of claim 34, wherein the particle is a cell on whose wall a fusion protein comprising the antigen is attached via conjugation.

50. The composition according to claim 49, wherein the antigen is attached via chemical conjugation or protein-mediated site-specific conjugation.

51. The composition according to claim 49, wherein the cell is a yeast cell of strain SWH100.

52. The composition according to claim 51, wherein the host cell is non-viable.

53. The composition according to claim 49, wherein the fusion protein comprises a fusion of a maltose-binding protein, SNAP-tag, 4 repeats of Cathepsin S recognition site EKARVLAEAA, and NY-ESO-1 as the antigen.

54. The composition according to claim 53, wherein the fusion protein comprises an amino acid sequence of SEQ ID NO:1.

55. The composition according to claim 34, wherein the particle is a pharmaceutically acceptable preparation of fungal or bacterial cell wall.

56. The composition according to claim 55, wherein the particle is zymosan or a yeast cell wall preparation.

57. The composition according to claim 34, wherein the particle comprises polymer beads, inorganic particles, micelles or colloidal complexes.

58. The composition according to claim 57, wherein the polymer beads comprises latex beads, poly(lactic-co-glycolic acid) beads, polystyrene beads, or chitosan beads.

59. The composition according to claim 57, wherein the inorganic particles are selected from the group consisting of iron oxide particles, glass beads, silica beads, gold particles, and Quantum Dots™.

60. The composition according to claim 57, wherein the particles comprise Immune-stimulating complexes (ISCOMs).

61. The composition according to claim 57, wherein the particles comprises liposomes.

62. The composition of claim 34, wherein a significant proportion of the antigen is released from the particle within about 20 minutes after phagocytosis.

63. A method for treating a population of cells, a cultured tissue, a cultured organ, or an animal in need thereof, the method comprising contacting said population of cells, cultured tissue or cultured organ of an animal with an pharmaceutical composition comprising a composition of claim 34 and a pharmaceutically acceptable excipient.

64. The method according to claim 63, further comprising transferring the treated population of cells, cultured tissue or cultured organ back to an animal.


SEQUENCE LISTING (29 sequences)

SEQ ID NO: 1 - protein, 802 aa, Artificial Sequence
  Description of Artificial Sequence Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGEFGGGGSASEKARVLAEAASEKARVLAEAASEKARVLAEAASEKARVLAEAASGGGSMQAEGRGTGGSTGDADGPGGPGIPDGPGGNAGGPGEAGATGGRGPRGAGAARASGPGGGAPRGPHGGAASGLNGSSRSGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 2 - protein, 645 aa, Artificial Sequence
  Description of Artificial Sequence Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGEFGGGGSASEKARVLAEAASEKARVLAEAASEKARVLAEAASEKARVLAEAASARNLVPMVATVQGQNGSEQKLISEEDL

SEQ ID NO: 3 - protein, 10 aa, Artificial Sequence
  Description of Artificial Sequence Synthetic peptide
EKARVLAEAA

SEQ ID NO: 4 - protein, 9 aa, Cytomegalovirus
NLVPMVATV

SEQ ID NO: 5 - protein, 15 aa, Artificial Sequence
  Description of Artificial Sequence Synthetic peptide
ARNLVPMVATVQGQN

SEQ ID NO: 6 - DNA, 50 bp, Artificial Sequence
  Description of Artificial Sequence Synthetic oligonucleotide
tagcgctagaaatttggttccaatggttgctactgttcaaggtcaaaacg

SEQ ID NO: 7 - DNA, 50 bp, Artificial Sequence
  Description of Artificial Sequence Synthetic oligonucleotide
atccgttttgaccttgaacagtagcaaccattggaaccaaatttctagcg

SEQ ID NO: 8 - protein, 9 aa, Artificial Sequence
  Description of Artificial Sequence Synthetic peptide
EKARVLAEA

SEQ ID NO: 9 - DNA, 33 bp, Artificial Sequence
  Description of Artificial Sequence Synthetic oligonucleotide
ctagtgaaaaagctagagttttggctgaagctg

SEQ ID NO: 10 - DNA, 33 bp, Artificial Sequence
  Description of Artificial Sequence Synthetic oligonucleotide
ctagcagcttcagccaaaactctagctttttca

SEQ ID NO: 11 - protein, 7 aa, Artificial Sequence
  Description of Artificial Sequence Synthetic peptide
SSAESLK

SEQ ID NO: 12 - DNA, 27 bp, Artificial Sequence
  Description of Artificial Sequence Synthetic oligonucleotide
ctagttcttctgctgaatctttgaaag

SEQ ID NO: 13 - DNA, 27 bp, Artificial Sequence
  Description of Artificial Sequence Synthetic oligonucleotide
ctagctttcaaagattcagcagaagaa

SEQ ID NO: 14 - protein, 8 aa, Artificial Sequence
  Description of Artificial Sequence Synthetic peptide
NWVCAAKF

SEQ ID NO: 15 - DNA, 30 bp, Artificial Sequence
  Description of Artificial Sequence Synthetic oligonucleotide
ctagtaattgggtttgtgctgctaaatttg

SEQ ID NO: 16 - DNA, 30 bp, Artificial Sequence
  Description of Artificial Sequence Synthetic oligonucleotide
ctagcaaatttagcagcacaaacccaatta

SEQ ID NO: 17 - protein, 8 aa, Artificial Sequence
  Description of Artificial Sequence Synthetic peptide
GILQINSR

SEQ ID NO: 18 - DNA, 30 bp, Artificial Sequence
  Description of Artificial Sequence Synthetic oligonucleotide
ctagtggtattttgcagattaattctagag

SEQ ID NO: 19 - DNA, 30 bp, Artificial Sequence
  Description of Artificial Sequence Synthetic oligonucleotide
ctagctctagaattaatctgcaaaatacca

SEQ ID NO: 20 - protein, 8 aa, Artificial Sequence
  Description of Artificial Sequence Synthetic peptide
QWLGAPVP

SEQ ID NO: 21 - DNA, 30 bp, Artificial Sequence
  Description of Artificial Sequence Synthetic oligonucleotide
ctagtcaatggttgggtgctccagttccag

SEQ ID NO: 22 - DNA, 30 bp, Artificial Sequence
  Description of Artificial Sequence Synthetic oligonucleotide
ctagctggaactggagcacccaaccattga

SEQ ID NO: 23 - protein, 154 aa, Artificial Sequence
  Description of Artificial Sequence Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASARNLVPMVATVQGQNGSEQKLISEEDL

SEQ ID NO: 24 - protein, 198 aa, Artificial Sequence
  Description of Artificial Sequence Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASEKARVLAEAASEKARVLAEAASEKARVLAEAASEKARVLAEAASARNLVPMVATVQGQNGSEQKLISEEDL

SEQ ID NO: 25 - protein, 15 aa, Artificial Sequence
  Description of Artificial Sequence Synthetic peptide
GGGGSGGGGSGGGGS

SEQ ID NO: 26 - protein, 5 aa, Artificial Sequence
  Description of Artificial Sequence Synthetic peptide
LPXTG

SEQ ID NO: 27 - protein, 5 aa, Artificial Sequence
  Description of Artificial Sequence Synthetic peptide
LPETG

SEQ ID NO: 28 - protein, 4 aa, Artificial Sequence
  Description of Artificial Sequence Synthetic peptide
MQAE

SEQ ID NO: 29 - protein, 5 aa, Artificial Sequence
  Description of Artificial Sequence Synthetic peptide
GGGGS